(19) United States
(12) Patent Application Publication
(10) Pub. No.: US 2024/0181137 A1
(43) Pub. Date: Jun. 6, 2024

(54) Superporous Gel Matrix for Encapsulation of Cells

(71) Applicant: The Regents of the University of California, Oakland, CA (US)

(72) Inventors: Charles Blaha, San Francisco, CA (US); Shuvo Roy, San Francisco, CA (US); Pujita Munnangi, San Francisco, CA (US); Ana Santandreu, San Francisco, CA (US); Rebecca Shaheen, San Francisco, CA (US)

(21) Appl. No.: 18/562,498

(22) PCT Filed: May 27, 2022
(86) PCT No.: PCT/US2022/031355

§ 371 Date: Nov. 20, 2023

Related U.S. Application Data
(60) Provisional application No. 63/194,000, filed on May 27, 2021.

(51) Int. Cl. A61L 27/52; A61L 27/20; A61L 27/36; A61L 27/56

(52) U.S. Cl. CPC A61L 27/52; A61L 27/20; A61L 27/3604; A61L 27/56; A61L 2400/12

(57) ABSTRACT

A biocompatible gel matrix that is produced from an emulsion comprising a water-soluble material capable of forming a gel and a biocompatible hydrophobic substance is provided. In certain aspects. the biocompatible gel matrix of the present disclosure may include a plurality of microchannels and a plurality of nanochannels, wherein the plurality of microchannels and the plurality of nanochannels are not patterned microchannels and nanochannels: and a plurality of cells, wherein the cells are adjacent the plurality of microchannels and wherein a majority of the plurality of cells are within a distance of 50 microns or less from at least one of the plurality of microchannels. wherein the plurality of microchannels have a width of 5-500 microns and the plurality of nanochannels have a width of 1 nm-500 nm. Methods of using the matrix and methods of making the matrix are also provided.

### CROSS-REFERENCE TO RELATED APPLICATIONS

This application claims priority to U.S. Provisional Application No. 63/194,000 filed May 27, 2021, which is herein incorporated by reference in its entirety.

### INTRODUCTION

Type 1 diabetes (T1D) results from autoimmune destruction of the insulin-producing B-cells within the pancreatic islets of Langerhans. Islet transplantation by direct infusion of cadaveric islets into the portal vein of the recipient's liver offers a non-invasive cure for patients with T1D mellitus 1. However, donor availability, poor engraftment, and side effects from global immunosuppression remain as obstacles for wider application of this approach. Moreover, up to 60% of the infused islets become nonviable within a few days after surgical delivery and the long-term insulin independence is frequently lost by 5 years of transplantation. The activation of innate and the adaptive immune responses are among the main causes of islet graft failure. The idea of encapsulating islets has generated tremendous interest. However, there is a need for improved devices and methods for providing encapsulated islets that maintain function and are protected from the patient's immune system.

### SUMMARY

A biocompatible gel matrix that is produced from an emulsion comprising a water-soluble material capable of forming a gel and a biocompatible hydrophobic substance is provided. Use of the biocompatible hydrophobic substance allows for generation of a biocompatible gel matrix that is non-toxic to cells and comprises microchannels that can support flow of nutrients to the cells. The matrix also comprises nanochannels that support diffusion of nutrient to the cells.

In certain aspects, the biocompatible gel matrix of the present disclosure may include a plurality of microchannels and a plurality of nanochannels, wherein the plurality of microchannels and the plurality of nanochannels are not patterned microchannels and nanochannels; and a plurality of cells, wherein the cells are adjacent the plurality of microchannels and wherein a majority of the plurality of cells are within a distance of 50 microns or less from at least one of the plurality of microchannels, wherein the plurality of microchannels have a width of 5-500 microns, e.g., 5-100 microns or 5-50 microns and the plurality of nanochannels have a width of 1 nm-500 nm.

In certain aspects, an emulsion for producing the biocompatible gel matrix is provided. The emulsion may be an oil-in-water emulsion comprising water-soluble material capable of forming a gel and a biocompatible hydrophobic substance, and optionally a surfactant. The emulsion may further include live cells. Upon cooling of the emulsion, a matrix is formed where the matrix comprises microchannels and nanochannels as described herein.

In certain aspects, the gel matrix is composed of a water-soluble material agarose, e.g., ultra-low gelling agarose. Examples of low gelling agarose include Type IX agarose, e.g., Type IX-A agarose. In certain aspects, the gel matrix is composed of a water-soluble material such as collagen, gelatin, polyethylene glycol, alginate, cellulose, PCL, or dextran.

In certain aspects, the gel matrix is in form of a planar scaffold, a cylinder, a sphere, or fibers. In certain aspects, the gel matrix comprises a volume of at least 1 cm³ to about 10,000 cm³. In certain aspects, the gel matrix comprises a surface area in the range of 1 cm²-1000 cm² or 15 cm²-30 cm².

In certain aspects, the gel matrix comprises at least 100 cells. In certain aspects, the cells may be uniformly dispersed in the matrix. In certain aspects, the cells may be single cells or a cluster of cells. In certain aspects, the cells are insulin producing cells. In certain aspects, the insulin producing cells are derived from differentiation of stem cells. In certain aspects, the insulin producing cells are pancreatic cells isolated from pancreatic islets. In certain aspects, the insulin producing cells are in islets isolated from pancreas and the islets are encapsulated in the matrix. In certain aspects, the islets each comprises about 1000 cells. In certain aspects, each islet has a diameter of about 100 microns. In certain aspects, the insulin producing cells are in stem-cell-derived enriched β-clusters (eBCs). In certain aspects, each eBC comprises about 1000 cells. In certain aspects, each eBC has a diameter of about 100 microns.

In certain aspects, the microchannels allow flow of nutrients to the plurality of cells and wherein at least 80% of the plurality of cells encapsulated in the matrix are viable for at least 1 day.

In certain aspects, the microchannels allow flow of nutrients to the plurality of cells and wherein at least 80% of the plurality of cells encapsulated in the matrix are viable for up to 1 month.

In certain aspects, the microchannels allow flow of nutrients to the plurality of cells and wherein at least 80% of the plurality of cells encapsulated in the matrix are viable and functional for at least 1 day.

In certain aspects, the microchannels allow flow of nutrients to the plurality of cells and wherein at least 80% of the plurality of cells encapsulated in the matrix are viable and functional for up to 1 month.

In certain aspects, the cells are insulin producing cells and function of the cells is assessed by exposing the cells to glucose and measuring insulin production. In certain aspects, the cells are exposed to insulin by flowing blood through the matrix.

In certain aspects, a bioartificial ultrafiltration device comprising a planar scaffold comprising the biocompatible gel matrix as disclosed herein is provided. The device may include a first semipermeable ultrafiltration membrane disposed on a first surface of the planar scaffold; a first compartment adjacent to the first surface of the planar scaffold and in fluidic communication with the planar scaffold via the first semipermeable ultrafiltration membrane and comprising an inlet and an outlet; and a second compartment adjacent to the second surface of the planar scaffold and comprising an outlet, wherein the first semipermeable ultrafiltration membrane comprises a plurality of pores having a width in the range of 5 nm-5 micron, wherein the first semipermeable ultrafiltration membrane allows transport of ultrafiltrate from the first compartment to the matrix and wherein the ultrafiltrate traverses through the matrix into the second compartment.

In certain aspects, the device further comprises a second semipermeable ultrafiltration membrane disposed on the second surface of the planar scaffold and wherein the ultrafiltrate traverses from the plurality of microchannels across the second semipermeable ultrafiltration membrane into the second compartment.

In certain aspects, the second semipermeable ultrafiltration membrane comprises a plurality of pores having a width in the range of 5 nm-5 micron. In certain aspects, the first and second semipermeable ultrafiltration membranes comprise a plurality of pores having a width in the range the range of 0.1 microns-2 microns, 0.2 microns-0.5 microns, 20 nm-2 microns, or 20 nm-50 nm.

In certain aspects, the second semipermeable ultrafiltration membrane comprises a plurality of pores having a width larger than the width of the plurality of pores in the first semipermeable ultrafiltration membrane.

In certain aspects, the inlet of the first compartment is attachable to a tubing for connection to a blood vessel of a subject, optionally, wherein the blood vessel is an artery of the subject. In certain aspects, the outlet of the first compartment is attachable to a tubing for connection to a blood vessel of a subject, optionally, wherein the blood vessel is a vein of the subject or to an artery of the subject. In certain aspects, the artery connected to the outlet is the same artery as connected to the inlet. In certain aspects, the outlet of the second compartment is attachable to a tubing for connection to (i) a blood vessel of a subject, and optionally provides the ultrafiltrate to one or more blood vessels of the subject, (ii) one or more veins of the subject, (iii) one or more arteries of the subject; and/or (iv) to an analyte analysis device.

In certain aspects, the thickness of the first semipermeable ultrafiltration membrane is in the range of 0.1 micron-100 micron or 0.5 micron-10 micron.

In certain aspects, the surface of the first and/or the second surface of the planar scaffold is in the range of 1 cm²-1000 cm², 1 cm²-100 cm², 10 cm²-100 cm², or 15 cm^{2 l -}30 cm². In certain aspects, the surface area of the first semipermeable ultrafiltration membrane is in the range of 1 cm²-100 cm² or 15 cm²-30 cm².

In certain aspects, the plurality of pores are circular in shape and wherein the width refers to diameter of the pores. In certain aspects, the plurality of pores are slit-shaped. In certain aspects, the plurality of pores are slit-shaped and wherein the width of the pores is 5 nm-500 nm, e.g., 5 nm-300 nm, 5 nm-200 nm, or 5 nm-100 nm. In certain aspects, the plurality of pores are slit-shaped and wherein the length of the pores is in the range of 0.1 micron-5 micron. In certain aspects, the plurality of pores are slit-shaped and wherein the length of the pores is in the range of 1 μm-3 μm and the width of the pores is 5 nm-100 nm. In certain aspects, the cells in the device are autologous to the subject, are xenogenic to the subject, or are allogenic to the subject.

In certain aspects, a bioartificial ultrafiltration device comprising a planar scaffold comprising the matrix as disclosed herein is provided. The device includes a first semipermeable ultrafiltration membrane, as disclosed herein, disposed on a first surface and a second semipermeable ultrafiltration membrane, as disclosed herein, disposed on a second surface of the planar scaffold; a first compartment comprising a first inlet and a first outlet, wherein the first compartment is adjacent to the first surface of the planar scaffold; a second compartment comprising a second inlet and a second outlet, wherein the second compartment is adjacent to the second surface of the planar scaffold, wherein the first inlet is configured for connection to an artery of a subject and the first outlet is connected to the second inlet of the second compartment, wherein the second outlet of the second compartment is configured for connection to a vein of the subject, where the semipermeable ultrafiltration membranes comprise a plurality of pores having a width in the range of 5nm-5 micron, wherein the first semipermeable ultrafiltration membrane allows transport of ultrafiltrate from the first compartment to the scaffold and the second semipermeable ultrafiltration membrane allows transport of the ultrafiltrate from the plurality of microchannels in the scaffold into the second compartment. In certain aspects, the cells in the device are autologous to the subject, are xenogenic to the subject, or are allogenic to the subject. In certain aspects, the plurality of pores in the second semipermeable ultrafiltration membrane have a width larger than the width of the plurality of pores in the first semipermeable ultrafiltration membrane or wherein the plurality of pores in the second semipermeable ultrafiltration membrane have a width smaller than the width of the plurality of pores in the first semipermeable ultrafiltration membrane.

A method for providing a bioartificial ultrafiltration device comprising cells to a subject in need thereof is disclosed. The method may include connecting the bioartificial ultrafiltration device as disclosed here to the subject, wherein the connecting comprises connecting the inlet of the first compartment to an artery of the subject and connecting the outlet of the first compartment to a blood vessel of the subject; and connecting the outlet of the second compartment to a blood vessel or a body cavity of the subject; or connecting the outlet of the second compartment to an analyte analysis device.

A method for providing a bioartificial ultrafiltration device comprising cells to a subject in need thereof is provided. The method may include connecting the bioartificial ultrafiltration device as disclosed here to the subject, wherein the connecting comprises connecting the first inlet to an artery of a subject; and connecting the second outlet to a vein of the subject.

In certain aspects, the method comprises providing insulin to the subject and wherein the cells comprise insulin producing cells. In certain aspects, connecting the bioartificial device to the subject in need thereof results in increased viability of the cells in the scaffold. In certain aspects, the ultrafiltrate comprises one or more of glucose and oxygen. In certain aspects, the ultrafiltrate comprises one or more of glucose and oxygen and wherein the insulin producing cells excrete insulin in response to presence of glucose in the ultrafiltrate and wherein the plurality of microchannels transport the insulin to the second compartment.

In certain aspects, the excreted insulin is transported to the plurality of microchannels in the scaffold. In certain aspects, the semipermeable ultrafiltration membranes prevent the passage of immune system components into the scaffold. In certain aspects, the semipermeable ultrafiltration membranes prevents passage of antibodies into the scaffold. In certain aspects, the semipermeable ultrafiltration membranes prevents passage of cytokines into the scaffold. In certain aspects, the semipermeable ultrafiltration membranes prevents passage of TNF-α, IFN-γ, and/or IL-1β into the scaffold.

In certain aspects, a method of making the biocompatible gel matrix disclosed herein is provided. The biocompatible gel matrix may be generated from agarose, gelatin, polyethylene glycol, polycaprolactone (PCL), collagen, alginate, dextran, or cellulose and includes a plurality of microchannels and a plurality of nanochannels, wherein the plurality of microchannels and the plurality of nanochannels are not patterned microchannels and nanochannels and wherein the plurality of microchannels have a width of 5-500 microns, 5-100 microns, or 5-50 microns and the plurality of nanochannels have a width of 1 nm-500 nm. In certain aspects, the method comprises dissolving agarose, gelatin, polyethylene glycol, PCL, collagen, alginate, dextran, or cellulose in an aqueous solution; adding a biocompatible hydrophobic substance and a surfactant to the aqueous solution; mixing the aqueous solution under conditions sufficient for generation of an emulsion comprising the dissolved agarose, gelatin, polyethylene glycol, PCL, collagen, alginate, dextran, or cellulose, biocompatible hydrophobic substance and surfactant; and generating the matrix by placing the emulsion at a temperature sufficient to allow gelation of the agarose, gelatin, polyethylene glycol, PCL, collagen, alginate, dextran, or cellulose, thereby creating the matrix. In certain aspects, the method may further include adding cells to the emulsion prior to the step of generating the matrix. In certain aspects, generating the matrix comprises casting the emulsion in a mold comprising a planar surface, thereby creating a planar scaffold. In certain aspects, the method further comprises disposing a first semipermeable ultrafiltration membrane on a first surface of the planar scaffold. In certain aspects, the method further comprises disposing a second semipermeable ultrafiltration membrane on a second surface of the planar scaffold. In certain aspects, the agarose is an ultra-low gelling agarose. In certain aspects, the agarose is present at a concentration of 1%-10% w/v, 2%-10% w/v, 2%-8% w/v, or 3%-6% w/v in the aqueous solution. In certain aspects, the dissolving the agarose comprises heating the aqueous solution to a temperature of about 37. C and stirring the solution at about 300 revolutions per minute (RPM). In certain aspects, generating the matrix comprises cooling the emulsion to a temperature at which the agarose, collagen, alginate, dextran, or cellulose forms a gel. In certain aspects, the water-immiscible reagent is perfluorodecalin (PFD).

### BRIEF DESCRIPTION OF THE DRAWINGS

FIGS. 1A-1D depict an SPA scaffold and intravascular bioartificial pancreas (iBAP) device assembly. FIG. 1A presents a schematic cross-sectional view of the SPA scaffold depicting the convective and diffusive pores in agarose. FIG. 1B presents an SPA scaffold within the islet chamber housing. FIG. 1C depicts an exploded view of the components of iBAP: flow path, silicon nanopore membrane (SNM), cell scaffold within the cell chamber, and polycarbonate (PC) backside and ultrafiltrate outlet. FIG. 1D presents an assembled iBAP used for in vitro testing.

FIG. 2 depicts a hydraulic permeability testing setup consisting of the iBAP, a peristaltic pump, and a pressure gauge. Ultrafiltration measurements were determined using a graduated syringe and a timer.

FIG. 3 shows that hydraulic permeability values (mean±SD, ** represents p<0.001) of scaffolds made with SPA and non-emulsified agarose were found to be statistically significant, F(1,12)-6986.

FIGS. 4A-4C depict a PFD droplet size analysis. FIG. 4A depicts representative DIC images of the 3% SPA scaffold that was used for droplet analysis. FIG. 4B presents a histogram showing the size distribution of PFD droplets representative PFD droplets represented as mean±SD for each scaffold. FIG. 4C depicts the relative droplet area of the scaffold calculated for each DIC image and then averaged. Lines represent the mean±SD for each scaffold and * represents p<0.05.

FIG. 5A-5B depict the degradation of the 3% SPA scaffolds. FIG. 5A show that the 28-day degradation study measured changes in weight and found no significant difference at the different time points. Data is shown as mean±SD. FIG. 5B shows representative images of the scaffold at 0, 7, 21, and 28 days of culture.

FIG. 6A-6B presents a histologic evaluation and viability of human islets and eBCs in the 3% traditional agarose and SPA scaffolds. FIG. 6A depicts representative images from H&E and viability staining of human islets and eBCs in traditional agarose and SPA scaffolds. FIG. 6B presents results for viability reported as mean+SD.

FIG. 7A-7B depicts an in vitro assessment of human islet and eBC insulin production in the optimized SPA formulation. Insulin production in the SPA scaffold is shown for both human islets (n=4) in FIG. 7A and eBCs (n=8) in FIG. 7B during the GSIS exposure to 5 mM of glucose (G5), 28 mM of glucose (G28), followed by a second G5 phase. Data represents mean±SD.

### DEFINITIONS

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are now described.

As used herein, the term “filtration” refers to a process of separating particulate matter from a fluid, such as air or a liquid, by passing the fluid carrier through a medium (e.g., a semipermeable membrane) that will not pass the particulates.

As used herein, the term “ultrafiltration” refers to subjecting a fluid to filtration, where the filtered material is very small; typically, the fluid comprises colloidal, dissolved solutes or very fine solid materials, and the filter is a microporous or nanoporous. The filter may be a membrane, such as, a semi-permeable membrane. The fluid to be filtered is referred to as the “feed fluid.” In certain embodiments, the feed fluid may be arterial blood. During ultrafiltration, the feed fluid is separated into a “permeate” or “filtrate” or “ultra-filtrate,” which has been filtered through the filter, and a “retentate,” which is that part of the feed fluid which did not get filtered through the membrane.

As used herein the terms “subject” or “patient” refers to a mammal, such as, a primate (e.g., humans or non-human primates), a bovine, an equine, a porcine, a canine, a feline, or a rodent. In certain embodiments, the subject or patient may be a human. In certain embodiments, the subject or patient may be pre-diabetic or may have diabetes, such as, type 1 diabetes (T1D) or type 2 diabetes. The terms “subject” and “patient” are used interchangeably herein.

As used herein, the terms “treat,” “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a subject, particularly in a human, and includes: (a) preventing the disease from occurring in a subject which may be predisposed to the disease but has not yet been diagnosed as having it; (b) inhibiting the disease, i.e., arresting its development; and (c) relieving the disease, e.g., causing regression of the disease, e.g., to completely or partially remove symptoms of the disease.

As used herein, the terms “layer”, “film”, or “membrane” and plurals thereof as used in the context of a device of the present disclosure refer to an individual layer of the device that may be formed from a silicon membrane, silicon nitride, silica, atomically thin membrane such as graphene, silicon, silicene, molybdenum disulfide (MoS₂), etc., or a combination thereof or a polymer. The “layer”, “film”, or “membrane” used to manufacture a porous layer of the present disclosure is typically porous and can be nanoporous or microporous. The phrases “nanoporous layer,” “nanopore layer,” “nanoporous membrane,” “nanopore membrane,” “nanoporous film,” and “nanopore film” are used interchangeably and all refer to a polymer layer in which nanopores have been created. A nanoporous layer may include a frame for supporting the layer. The phrases “microporous layer,” “micropore layer,” “microporous membrane,” “micropore membrane,” “microporous film,” and “micropore film” are used interchangeably and all refer to a polymer layer in which micropores have been created. A microporous layer may include a frame for supporting the layer.

As used herein, the term “encapsulated” as used in the context of cells disposed in a matrix as described herein refers to cells that are surrounded by the matrix as opposed to being present in the microchannels in the matrix. Encapsulated cells are immobilized in the matrix such that they do not move significantly move in the matrix. Encapsulated cells receive nutrients via flow of solution in the microchannels in the matrix surrounding the cells. Encapsulated cells receive nutrients via diffusion of nutrient in the nanochannels in the matrix surrounding the cells.

As used herein the term “biocompatible” refers to a material, matrix, or device that is not significantly toxic to cells, e.g., mammalian cells.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “channels” includes a plurality of such channels and reference to “the agarose-cell region” includes reference to one or more agarose-cell regions and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

As summarized above, a biocompatible gel matrix that is produced from an emulsion comprising a water-soluble material capable of forming a gel and a biocompatible hydrophobic substance is provided. Use of the biocompatible hydrophobic substance allows for generation of a biocompatible gel matrix that is non-toxic to cells and comprises microchannels that can support flow of nutrients to the cells. The matrix also comprises nanochannels that support diffusion of nutrient to the cells. In absence of use of a hydrophobic substance to generate an emulsion, a biocompatible gel matrix formed from a water-soluble material capable of forming a gel does not include microchannels. Absence of microchannels significantly reduces hydraulic permeability of the matrix rendering it unsuitable for use to support live cells and for its use as a device to provide cells for various applications. Use of a biocompatible hydrophobic substance allows encapsulation of live cells in the matrix.

In certain aspects, the biocompatible gel matrix of the present disclosure may include a plurality of microchannels and a plurality of nanochannels, wherein the plurality of microchannels and the plurality of nanochannels are not patterned microchannels and nanochannels; and a plurality of cells, wherein the cells are adjacent the plurality of microchannels and wherein a majority of the plurality of cells are within a distance of 50 microns or less from at least one of the plurality of microchannels, wherein the plurality of microchannels have a width of 5-500 microns, 5-100 microns, or 5-50 microns and the plurality of nanochannels have a width of 1 nm-500 nm. As a result of the process of forming the microchannels, the microchannels are not straight channels and/or uniformly placed channels such as those obtained from patterning.

In certain aspects, the biocompatible gel matrix of the present disclosure does not include laser-cut voids or voids introduced by solidifying the matrix around hollow tubes in order to include through-channels in the matrix.

In certain aspects, a majority of the plurality of cells are within a distance of 40 microns or less, 30 microns or less, 20 microns or less, 10 microns or less, 5 microns or less, 1 microns or less, or immediately adjacent at least one of the plurality of microchannels. In certain aspects, a majority of the plurality of cells 55% or more, 60% or more, 65% or more, 70% or more, 75% or more, 80% or more, or 85% or more.

In certain aspects, the plurality of microchannels have a width of 5-500 microns, 5-100 microns, or 5-50 microns. In certain aspects, the plurality of microchannels are circular and the width refers to an average diameter of the microchannels. Thus, for example, the diameter of a microchannel may be in the range of 5-100 microns. In certain aspects, the plurality of microchannels have a width of 5-30 microns, 5-20 microns, 10-30 microns, or 20-30 microns.

In certain aspects, the plurality of nanochannels have a width of 1 nm-500 nm. In certain aspects, the plurality of nanochannels are circular and the width refers to an average diameter of the nanochannels. Thus, for example, the diameter of a nanochannels may be in the range of 1 nm-500 nm, 1 nm-250 nm, 1 nm-200 nm, 1 nm-100 nm, 10 nm-100 nm, or 1 nm-50 nm.

The plurality of microchannels and nanochannels are not patterned and are not fabricated by using patterning techniques such as those used for generating channels from PCL or silicon carbide, etc.

In certain aspects, an emulsion for producing the biocompatible gel matrix is provided. The emulsion may be an oil-in-water emulsion comprising water-soluble material capable of forming a gel and a biocompatible hydrophobic substance, and optionally a surfactant. The emulsion may further include live cells. Upon cooling of the emulsion, a matrix is formed where the matrix comprises microchannels and nanochannels as described herein.

In certain aspects, the gel matrix is composed of a water-soluble material, such as, agarose, e.g., ultra-low gelling agarose. In certain aspects, the gel matrix is composed of a water-soluble material such as collagen, gelatin, polyethylene glycol, PCL, alginate, cellulose, or dextran.

In certain aspects, the gel matrix is in shape of a planar scaffold, a cylinder, a sphere, or fibers. For example, the emulsion may be transferred to a mold of any desirable shape and cooled and removed from the mold. In certain aspects, the mold may be formed from a semipermeable ultrafiltration membrane. In certain aspects, a planar scaffold refers to a shape that has a cuboid shape.

In certain aspects, the gel matrix comprises a volume of at least 1 cm³ to about 10,000 cm³. For example, the gel matrix may have a volume of 1 cm³-1000 cm³, 10 cm³ to about 10,000 cm³, or 10 cm³ to about 1000 cm³. In certain aspects, the gel matrix comprises a surface area in the range of 1 cm²-1000 cm², e.g., 1 cm²-50 cm², 10 cm²-100 cm², 10 cm²-50 cm², or 15 cm²-30 cm².

In certain aspects, the gel matrix comprises at least 100 cells, e.g., 1000 cells, 10,000 cells, 100,000 cells, 10⁶ cells, 10⁸ cells, 10¹⁰ cells, 10¹² cells, 10¹⁴ cells, or more. In certain aspects, the cells may be uniformly dispersed in the matrix. In certain aspects, the cells may be single cells or a cluster of cells. In certain aspects, the cells are insulin producing cells. In certain aspects, the insulin producing cells are derived from differentiation of stem cells. In certain aspects, the insulin producing cells are pancreatic cells isolated from pancreatic islets. In certain aspects, the insulin producing cells are in islets isolated from pancreas and the islets are encapsulated in the matrix. In certain aspects, the islets each comprises about 1000 cells, e.g., 500-5000 cells or 800-1500 cells. In certain aspects, each islet has a diameter of about 100 microns, e.g., 50-200 μm, 50-150 μm, 80-200 μm, 80-150 μm, or 90-125 μm. In certain aspects, the insulin producing cells are in stem-cell-derived enriched β-clusters (eBCs). In certain aspects, each eBC comprises about 1000 cells, e.g., 500-5000 cells or 800-1500 cells. In certain aspects, each eBC has a diameter of about 100 microns, e.g., 50-200 μm, 50-150 μm, 80-200 μm, 80-150 μm, or 90-125 μm.

In certain aspects, the microchannels allow flow of nutrients to the plurality of cells and wherein at least 80%, at least 85%, or at least 90% of the plurality of cells encapsulated in the matrix are viable for at least 1 day, at least 10 days, at least 30 days, at least 3 months, at least 6 months, or more.

In certain aspects, the microchannels allow flow of nutrients to the plurality of cells and wherein at least 80%, at least 85%, or at least 90% of the plurality of cells encapsulated in the matrix are viable for up to 1 month, up to 2 months, up to 3 months, or up to 6 months.

In certain aspects, the microchannels allow flow of nutrients to the plurality of cells and wherein at least 80%, at least 85%, or at least 90% of the plurality of cells encapsulated in the matrix are viable and functional for at least 1 day, at least 10 days, at least 30 days, at least 3 months, at least 6 months, or more.

In certain aspects, the microchannels allow flow of nutrients to the plurality of cells and wherein at least 80%, at least 85%, or at least 90% of the plurality of cells encapsulated in the matrix are viable and functional for up to 1 month, up to 2 months, up to 3 months, or up to 6 months.

In certain aspects, the cells are insulin producing cells and function of the cells is assessed by exposing the cells to glucose and measuring insulin production. In certain aspects, the cells are exposed to insulin by flowing blood through the matrix.

In certain aspects, the biocompatible gel matrix has a hydraulic permeability that is higher than a biocompatible gel matrix formed without forming an emulsion since a biocompatible gel matrix formed without first forming an emulsion does not include significant number of microchannels. As explained in the Examples section, the microchannels are formed from bubbles formed by use of a hydrophobic substance which bubbles coalesce during cooling of the emulsion during formation of the matrix.

In certain aspects, the gel matrix comprises a water-soluble substance capable for forming a gel upon cooling. Examples of such substances include agarose and collagen. In certain aspects, the gel matrix does not comprise alginate, alginate derivative, gelatin, collagen, fibrin, hyaluronic acid, matrigel, natural polysaccharide, synthetic polysaccharide, polyamino acid, polyester, polyanhydride, polyphosphazine, poly(vinyl alcohol), poly(alkylene oxide), modified styrene polymer, pluronic polyol, polyoxamer, poly(uronic acid), or poly(vinylpyrrolidone) polymer polylactic acid, polyglycolic acid, PLGA polymers, polyesters, poly(allylamines)(PAM), poly(acrylates), polyethylene glycol, fibrin, PCL, and poly (methyl methacrylate) and copolymers or graft copolymers of any of the above.

As used herein, the term gel refers to a matrix comprising water and a water-soluble substance that forms the gel or matrix after being dissolved in an aqueous solution and upon cooling below a certain temperature. The water-soluble substance is hydrophilic and swellable and forms crosslinks to create the gel. The gel can be dissolved after forming, by exposing the gel to a temperature higher than the temperature at which the water-soluble substance sets to form the gel.

Cells that can be included in the matrices, scaffolds, and devices described herein include but are not limited to, bone marrow cells; mesenchymal stem cells, stromal cells, pluripotent stem cells (e.g., induced pluripotent stem cells or embryonic stem cells), blood vessel cells, precursor cells derived from adipose tissue, bone marrow derived progenitor cells, intestinal cells, islets, Sertoli cells, beta cells, progenitors of islets, progenitors of beta cells, peripheral blood progenitor cells, stem cells isolated from adult tissue, retinal progenitor cells, cardiac progenitor cells, osteoprogenitor cells, neuronal progenitor cells, and genetically transformed cells, or a combination thereof. The population of cells may be from the subject (autologous cells), from another donor (allogeneic cells) or from other species (xenogeneic cells). The cells can be introduced into the matrix and the matrix may be immediately (within a day) implanted into a subject or the cells may be cultured for longer period, e.g., greater than one day, to allow for cell proliferation prior to implantation.

In certain embodiments, the populations of cells in the matrix are stem cells. In certain embodiments, the population of cells in the matrix are pancreatic progenitor cells. In certain embodiments, the population of cells in the matrix are pancreatic cells isolated from islets of pancreas. In certain embodiments, the population of cells in the matrix are islets isolated from pancreas. In certain embodiments, the population of cells in the matrix may be in the form of a piece of tissue, such as, islet of Langerhans, which may have been isolated from the subject receiving the device or from another subject.

In certain embodiments, the devices disclosed herein may be used to treat a person having diabetes, such as, type 1 diabetes. The device may include pancreatic islet cells or may include stem cells that are capable of differentiating into insulin producing pancreatic cells. In certain embodiments, pluripotent stem cells (PSCs) may be differentiated into insulin producing pancreatic cells inside the device and then the bioartificial device containing the differentiated insulin producing pancreatic cells is placed in the subject (e.g., in the omentum, adjacent to pancreas or liver, adjacent to kidney, lung, or heart, or subdermally, e.g., in arm or abdomen). In some case, the device may include PSCs and the device may be implanted adjacent the pancreas or liver of the subject.

In certain aspects, a bioartificial ultrafiltration device comprising a planar scaffold comprising the biocompatible gel matrix as disclosed herein is provided. The device may include a first semipermeable ultrafiltration membrane disposed on a first surface of the planar scaffold; a first compartment adjacent to the first surface of the planar scaffold and in fluidic communication with the planar scaffold via the first semipermeable ultrafiltration membrane and comprising an inlet and an outlet; and a second compartment adjacent to the second surface of the planar scaffold and comprising an outlet, wherein the first semipermeable ultrafiltration membrane comprises a plurality of pores having a width in the range of 5 nm-5 micron, wherein the first semipermeable ultrafiltration membrane allows transport of ultrafiltrate from the first compartment to the matrix and wherein the ultrafiltrate traverses through the matrix into the second compartment.

In certain aspects, the device further comprises a second semipermeable ultrafiltration membrane disposed on the second surface of the planar scaffold and wherein the ultrafiltrate traverses from the plurality of microchannels across the second semipermeable ultrafiltration membrane into the second compartment.

In certain aspects, the second semipermeable ultrafiltration membrane comprises a plurality of pores having a width in the range of 5 nm-5 micron. In certain aspects, the first and second semipermeable ultrafiltration membranes comprise a plurality of pores having a width in the range the range of 0.1 microns-2 microns, 0.2 microns-0.5 microns, 20 nm-2 microns, or 20 nm-50 nm.

In certain aspects, the second semipermeable ultrafiltration membrane comprises a plurality of pores having a width larger than the width of the plurality of pores in the first semipermeable ultrafiltration membrane.

In certain aspects, the inlet of the first compartment is attachable to a tubing for connection to a blood vessel of a subject, optionally, wherein the blood vessel is an artery of the subject. In certain aspects, the outlet of the first compartment is attachable to a tubing for connection to a blood vessel of a subject, optionally, wherein the blood vessel is a vein of the subject or to an artery of the subject. In certain aspects, the artery connected to the outlet is the same artery as connected to the inlet. In certain aspects, the outlet of the second compartment is attachable to a tubing for connection to (i) a blood vessel of a subject, and optionally provides the ultrafiltrate to one or more blood vessels of the subject, (ii) one or more veins of the subject, (iii) one or more arteries of the subject; and/or (iv) to an analyte analysis device.

In certain aspects, the thickness of the first semipermeable ultrafiltration membrane is in the range of 0.1 micron-100 micron or 0.5 micron-10 micron.

In certain aspects, the surface of the first and/or the second surface of the planar scaffold is in the range of 1 cm²-100 cm² or 15 cm²-30 cm². In certain aspects, the surface area of the first semipermeable ultrafiltration membrane is in the range of 1 cm²-100 cm² or 15 cm²-30 cm².

In certain aspects, the plurality of pores are circular in shape and wherein the width refers to diameter of the pores. In certain aspects, the plurality of pores are slit-shaped. In certain aspects, the plurality of pores are slit-shaped and wherein the width of the pores is 5 nm-500 nm, 5 nm-400 nm, 5 nm-300 nm, 5 nm-200 nm, 5 nm-100 nm, or 5 nm-50 nm. In certain aspects, the plurality of pores are slit-shaped and wherein the length of the pores is in the range of 0.1 micron-5 micron. In certain aspects, the plurality of pores are slit-shaped and wherein the length of the pores is in the range of 1 μm-3 μm and the width of the pores is 5 nm-100 nm. In certain aspects, the cells in the device are autologous to the subject, are xenogenic to the subject, or are allogenic to the subject.

In certain aspects, a bioartificial ultrafiltration device comprising a planar scaffold comprising the matrix as disclosed herein is provided. The device includes a first semipermeable ultrafiltration membrane, as disclosed herein, disposed on a first surface and a second semipermeable ultrafiltration membrane, as disclosed herein, disposed on a second surface of the planar scaffold; a first compartment comprising a first inlet and a first outlet, wherein the first compartment is adjacent to the first surface of the planar scaffold; a second compartment comprising a second inlet and a second outlet, wherein the second compartment is adjacent to the second surface of the planar scaffold, wherein the first inlet is configured for connection to an artery of a subject and the first outlet is connected to the second inlet of the second compartment, wherein the second outlet of the second compartment is configured for connection to a vein of the subject, where the semipermeable ultrafiltration membranes comprise a plurality of pores having a width in the range of 5 nm-5 micron, wherein the first semipermeable ultrafiltration membrane allows transport of ultrafiltrate from the first compartment to the scaffold and the second semipermeable ultrafiltration membrane allows transport of the ultrafiltrate from the plurality of microchannels in the scaffold into the second compartment. In certain aspects, the cells in the device are autologous to the subject, are xenogenic to the subject, or are allogenic to the subject. In certain aspects, the plurality of pores in the second semipermeable ultrafiltration membrane have a width larger than the width of the plurality of pores in the first semipermeable ultrafiltration membrane or wherein the plurality of pores in the second semipermeable ultrafiltration membrane have a width smaller than the width of the plurality of pores in the first semipermeable ultrafiltration membrane.

In some cases, the first compartment into which the blood is introduced into the device may have a dimension suitable for facilitating ultrafiltration of the blood. For example, the first compartment may have a height of 100 micron-6 mm, e.g., 500 micron-4 mm, 1 mm-3 mm, or 2 mm-3mm.

In certain embodiments, the bioartificial device is dimensioned to fit in a body cavity of a subject. The device may be rectangular or cylindrical in shape. In certain case, the device may have a surface area of 50 cm² or less, such as 10-30 cm², 10-25 cm², 15-25 cm², 20-25 cm², 15-30 cm². In certain cases, the device may be rectangular and have a length of 3 cm-10 cm, a width of 1 cm-6 cm, and a height of 0.3 cm-2 cm, such as dimension (length×width×height) of 3 cm×1 cm×0.5 cm to 6 cm×4 cm×1 cm, e.g., 3 cm×1 cm×0.5 cm, 5 cm×2 cm×1 cm, or 6 cm×4 cm×1 cm.

As noted herein, the devices disclosed herein may maintain the transplanted cells in a functional and viable state for at least 1 month and up to a period of at least 2 months, 3 months, 4 months, 5 months, 6 months, 7 months, 8 months, 9 months, 10 months, 11 months, 1 year, 3 years, 5 years, 10 years, or up to 50 years, or longer, such as, 1 month-50 years, 1 year-25 years, 5 years-50 years, 5 years-25 years, 10 years-50 years, or 15 years-25 years.

In certain embodiments, the devices disclosed herein may be enclosed in a housing made from an inert material that does not degrade or foul when placed in a subject. Any material approved for medical devices placed in a subject may be utilized including but not limited to medical grade plastic, inert metals, such as, titanium, stainless steel, etc.

In certain embodiments, the bioartificial device comprises more than one semipermeable ultrafiltration membrane. In certain embodiments, the semipermeable ultrafiltration membrane is disposed on the first surface and the second surface of the planar scaffold. The semipermeable ultrafiltration membrane disposed on a first surface of the scaffold may be the same as the semipermeable ultrafiltration membrane disposed on the second surface of the scaffold or may be different. For example, the semipermeable ultrafiltration membrane adjacent to a compartment containing arterial blood may have smaller pores than the semipermeable ultrafiltration membrane adjacent a compartment containing the ultrafiltrate flowing through the channels in the matrix. In some cases, the semipermeable ultrafiltration membrane adjacent to a compartment containing arterial blood may have larger pores than the semipermeable ultrafiltration membrane adjacent a compartment containing the ultrafiltrate flowing through the channels in the matrix. In certain embodiments, the semipermeable membrane allows for filtration of an ultrafiltrate from the compartment containing arterial blood which ultrafiltrate is transported into the plurality of microchannels in the scaffold. The plurality of microchannels are adjacent the cells which provides for efficient exchange of molecules in the ultrafiltrate in the microchannels with the molecules released by the cells. These molecules diffuse in a concentration dependent manner between the lumen of the microchannels and the matrix surrounding the cells. For example, molecules, such as oxygen, glucose, lipids, vitamins, and minerals diffuse from the lumen of the channels into the matrix to the cells and molecules secreted by the cells such as urea, carbon dioxide, insulin are transported into the lumen of the microchannels. It is understood that in some embodiments, the diffusion and exchange of molecules in the ultrafiltrate may occur outside of the microchannels, such as, with the ultrafiltrate that does not enter the microchannels and permeates through the matrix, via, e.g., nanochannels.

In certain embodiments, the semipermeable ultrafiltration membrane is configured for filtration of biological fluids. In certain embodiments, the membrane comprises a plurality of nanopores, where the shapes and sizes of the pores are controlled. In certain embodiments, the membrane comprises a plurality of pores. In certain embodiments, the plurality of pores may be micropores and have a width in the range of 0.1 μm -5 μm, e.g., 0.1 μm-3 μm, 0.1 μm-0.5 μm, 0.5 μm-1 μm, 1 μm-1.5 μm, 1.5 μm-2 μm, 0.1 μm-1 μm, 0.1 μm-0.8 μm, 0.2 μm-0.7 μm, 0.2 μm-0.6 μm, 0.2 μm-0.5 μm. In certain embodiments, the plurality of pores may be nanopores and may have a width of 1 nm-500 nm, e.g., 1 nm-90 nm, 2 nm-50 nm, 3 nm-40 nm, 4 nm-50 nm, 4 nm-40 nm, 5 nm-50 nm, 5 nm-20 nm, 4 nm-20 nm, 7 nm-100 nm, 12 nm-20 nm, or 5 nm-10 nm. In certain embodiments, the plurality of pores are slit shaped and have a width as listed herein and have a length in the range of 1 μm-10 μm, e.g., 2 um-3 μm, 3 μm-4 μm, 4 μm-5 μm, 5 μm-6 μm, 6 μm-7 μm, 7 μm-8 μm, 8 μm-9 μm, or 9 μm-10 μm. In certain cases, the rectangular pores have a depth of 100-1000 nm, a width of 3 nm-50 nm and a length of 1 micron-5 micron, e.g., a width×length×depth of 5 nm-50 nm×1 micron-2 micron×200 nm-500nm.

In certain embodiments, the devices of the present disclosure include semipermeable ultrafiltration membranes having a dimension (length×width) of 6 mm×6 mm, 5 mm×5 mm, 7 mm×7 mm, 8 mm×8 mm, 9 mm×9 mm, 10 mm×10 mm, 10 cm×10 cm, e.g., 10 mm×10 mm to 10 cm×10 cm. In some embodiments, the semipermeable ultrafiltration membrane may be rectangular. In certain embodiments, the semipermeable ultrafiltration membrane has a surface area in the range of 0.5-100 cm², e.g., 30-100 cm², 10-30 cm², 15-30 cm², 15-20 cm², 20-25 cm², 25-30 cm², 0.5-10 cm², 0.75-5 cm², 0.75-3 cm², or 0.75-2 cm².

In certain embodiments, the devices disclosed herein may be substantially planar and may be dimensioned to have a surface area ranging from 20-100 cm² (on each planar side) and a thickness of 1 cm-3 cm. In certain embodiments, the devices disclosed herein may have a volume of up to 500 cm³, such as, 50-500 cm³, 100-500 cm³, 100-300 cm³, 100-150 cm³. In certain cases, the device may include a semipermeable membrane having a surface of 5-75 cm², e.g., 5-50 cm², 10-30 cm², or 15-30 cm². The size of pores in the membrane may be 10 nm-100 nm in width, such as, 10 nm-20 nm.

The semipermeable ultrafiltration membranes of the present disclosure include any membrane material suitable for use in filtering biological fluids, wherein the membranes are structurally capable of supporting formation of pores. Examples of suitable membrane materials are known in the art and are described herein.

In certain embodiments, the membrane material is synthetic, biological, and/or biocompatible (e.g., for use outside or inside the body). Materials include, but are not limited to, silicon, which is biocompatible, coated silicon materials, polysilicon, silicon carbide, ultrananocrystalline diamond, diamond-like carbond (DLC), silicon dioxide, PMMA, SU-8, and PTFE. Other possible materials include metals (for example, titanium), ceramics (for example, silica or silicon nitride), and polymers (such as polytetrafluorethylene, polymethylmethacrylate, polystyrenes and silicones). Materials for membranes can be found in, for example U.S. Patent Application Publication No. 20090131858, which is hereby incorporated by reference in its entirety.

A semipermeable ultrafiltration membrane of the present disclosure comprises a plurality of pores, where pore shapes include linear, square, rectangular (slit-shaped), circular, ovoid, elliptical, or other shapes. As used herein, width of a pore refers to the diameter where the pore is circular, ovoid or elliptical. In certain embodiments, the membrane comprises pores comprising a single shape or any combination of shapes. In certain embodiments, the sizes of pores are highly uniform. In certain embodiments, the pores are micromachined such that there is less than 20% size variability, less than 10% size variability, or less than 5% size variability between the dimensions of the slit-shaped pores. In certain embodiments, factors that determine appropriate pore size and shape include a balance between hydraulic permeability and solute permselectivity. In certain embodiments, the plurality of pores are slit-shaped pores which provide for optimum flux efficiency enabling efficient transport of molecules across the membrane. In certain embodiments, the membrane comprises slit-shaped nanopores. In certain embodiments, the semipermeable ultrafiltration membrane has approximately 10³-10⁸ rectangular slit-shaped nanopores (e.g., 10⁴-10⁸, or 10⁵-10⁷) for example on a membrane surface area of 1 cm², 0.5 cm², or 0.4 cm². In certain embodiments, the number of slit-shaped nanopores on the semipermeable ultrafiltration membrane is sufficient to allow the membrane to generate physiologically sufficient ultrafiltration volume at capillary perfusion pressure. In certain embodiments, the porosity of the semipermeable ultrafiltration membrane is approximately 1%-50%, e.g., 10%-50%, 20%-50%, or 20%-75%, etc.).

### Method of Using

A method for providing a bioartificial ultrafiltration device comprising cells to a subject in need thereof is disclosed. The method may include connecting the bioartificial ultrafiltration device as disclosed here to the subject, wherein the connecting comprises connecting the inlet of the first compartment to an artery of the subject and connecting the outlet of the first compartment to a blood vessel of the subject; and connecting the outlet of the second compartment to a blood vessel or a body cavity of the subject; or connecting the outlet of the second compartment to an analyte analysis device.

A method for providing a bioartificial ultrafiltration device comprising cells to a subject in need thereof is provided. The method may include connecting the bioartificial ultrafiltration device as disclosed here to the subject, wherein the connecting comprises connecting the first inlet to an artery of a subject; and connecting the second outlet to a vein of the subject.

In certain aspects, the method comprises providing insulin to the subject and wherein the cells comprise insulin producing cells. In certain aspects, connecting the bioartificial device to the subject in need thereof results in increased viability of the cells in the scaffold. In certain aspects, the ultrafiltrate comprises one or more of glucose and oxygen. In certain aspects, the ultrafiltrate comprises one or more of glucose and oxygen and wherein the insulin producing cells excrete insulin in response to presence of glucose in the ultrafiltrate and wherein the plurality of microchannels transport the insulin to the second compartment.

In certain aspects, the excreted insulin is transported to the plurality of microchannels in the scaffold. In certain aspects, the semipermeable ultrafiltration membranes prevent the passage of immune system components into the scaffold. In certain aspects, the semipermeable ultrafiltration membranes prevents passage of antibodies into the scaffold. In certain aspects, the semipermeable ultrafiltration membranes prevents passage of cytokines into the scaffold. In certain aspects, the semipermeable ultrafiltration membranes prevents passage of TNF-α, IFN-γ, and/or IL-1β into the scaffold. In certain embodiments, the bioartificial device of the present disclosure can reduce passage of TNF-α, IFN-γ, and/or IL-1β while permitting transport of nutrients from the blood of the subject to the cells in the device. In certain embodiments, the bioartificial device of the present disclosure can reduce passage of components of the immune system (e.g., immune cells, antibodies, cytokines, such as, TNF-α, IFN-γ, and/or IL-1β) by at least 50% (e.g., 60%-80%). In certain embodiments, the bioartificial device of the present disclosure having semipermeable ultrafiltration membranes with nanopores (e.g., The bioartificial device of the present disclosure are sized to house an effective number for cells within the bioartificial device for treatment of a subject in need thereof. For example, the subject may be suffering from a condition caused by lack of functional cells, e.g., wherein molecules typically secreted by functional cells are not secreted or are secreted at a level resulting in the condition. Providing functional cells within the bioartificial device of the present disclosure could alleviate the condition. Exemplary conditions include type 1 diabetes, Parkinson's disease, muscular dystrophy and the like.

The device may be transplanted into any suitable location in the body, such as, subcutaneously, intraperitoneally, or in the brain, spinal cord, pancreas, liver, uterus, skin, bladder, kidney, muscle and the like. The site of implantation may be selected based on the diseased/injured tissue that requires treatment. For treatment of a disease such as diabetes mellitus (DM), the device may be placed in a clinically convenient site such as the subcutaneous space or the omentum. The device may be connected to the vascular system of the subject as described herein. In some case, the device may be connected inline to a vascular graft. In some cases, the device may be connected to the subject to supply the ultrafiltrate to an artery, a vein, a body cavity (e.g., peritoneal cavity), or a combination thereof, of the subject. In some cases, the device may be connected to a catheter to supply the ultrafiltrate to a vein to which the catheter is connected.

The methods and devices disclosed herein can be used for both human clinical and veterinary applications. Thus, the subject or patient to whom the bioartificial device is administered can be a human or, in the case of veterinary applications, can be a laboratory, agricultural, domestic, or wild animal. The subject devices and methods can be applied to animals including, but not limited to, humans, laboratory animals such as monkeys and chimpanzees, domestic animals such as dogs and cats, agricultural animals such as cows, horses, pigs, sheep, goats, and wild animals in captivity such as bears, pandas, lions, tigers, leopards, elephants, zebras, giraffes, gorillas, dolphins, and whales.

In operation, blood is directed from a patient's vasculature (i.e. artery) into the inlet of the first compartment of the bioartificial device. Blood flows through the first compartment of the bioartificial device, and nutrients and small molecules from the blood are passed through the semipermeable ultrafiltration membrane, while large molecules, such as immunoglobulins and cytokines within the blood are prevented from coming in contact with the cells in the device. Nutrients and small molecules include, but are not limited to glucose, oxygen, and insulin. The small molecules and nutrients that pass through the semipermeable ultrafiltration membrane are filtered to form an ultrafiltrate which contacts the matrix of the device comprising the population of cells. In certain embodiments, the population of cells release insulin into the ultrafiltrate. The ultrafiltrate then passes through the ultrafiltrate channels of the matrix, which then passes through a second semipermeable ultrafiltration membrane. Optionally, the outlet of the second compartment can be configured to connect to a catheter. In certain embodiments, the catheter connects to the second vein.

The disclosed devices provide a high rate of ultrafiltration creating ultrafiltrate at the rate of 1-15 ml/min at physiological rate of blood flow.

### Method of Making

In certain aspects, a method of making the biocompatible gel matrix disclosed herein is provided. The biocompatible gel matrix may be generated from a water-soluble gel forming material such as agarose, collagen, alginate, dextran, or cellulose and includes a plurality of microchannels and a plurality of nanochannels, wherein the plurality of microchannels and the plurality of nanochannels are not patterned microchannels and nanochannels and wherein the plurality of microchannels have a width of 5-100 microns and the plurality of nanochannels have a width of 1 nm-500 nm.

In certain aspects, the method comprises dissolving agarose, gelatin, polyethylene glycol, PCL, collagen, alginate, dextran, or cellulose in an aqueous solution; adding a biocompatible hydrophobic substance and a surfactant to the aqueous solution; mixing the aqueous solution under conditions sufficient for generation of an emulsion comprising the dissolved agarose, gelatin, polyethylene glycol, collagen, alginate, dextran, or cellulose, biocompatible hydrophobic substance and surfactant; and generating the matrix by placing the emulsion at a temperature sufficient to cause gelation of the agarose, gelatin, polyethylene glycol, PCL, collagen, alginate, dextran, or cellulose, thereby creating the matrix. In certain aspects, the method may further include adding cells to the emulsion prior to the step of generating the matrix.

In certain aspects, dissolving the water-soluble gel forming material in an aqueous solution may involve mixing, e.g., stirring, a solution of the water-soluble gel forming material and a buffer or a balanced salt solution. In certain aspects, dissolving may also involve applying heat to the solution prior to, during, and/or after the mixing step.

In certain aspects, adding a biocompatible hydrophobic substance and a surfactant to the aqueous solution and mixing may include a step of mixing a solution of the water-soluble gel forming material and the biocompatible hydrophobic substance and heating the solution of the to a temperature of 37. C and stirring at about 300 RPM (e.g., 100-500 RPM) and adding a surfactant (e.g., Tween 80 or Tween 20) and stirring for a period of time. The solution may be stirred at a higher speed, e.g., higher than 500 RPM (e.g., higher than 600-1000RPM) to create an emulsion.

The step of generating the matrix by placing the emulsion at a temperature sufficient to allow gelation of the agarose, gelatin, polyethylene glycol, PCL, collagen, alginate, dextran, or cellulose, thereby creating the matrix may include placing the emulsion at room temperature (e.g., 25. C) and/or at 4. C for a period of time sufficient for gelation of the water-soluble gel forming material.

In certain aspects, generating the matrix comprises casting the emulsion in a mold comprising a planar surface, thereby creating a planar scaffold. In certain aspects, the method further comprises disposing a first semipermeable ultrafiltration membrane on a first surface of the planar scaffold. In certain aspects, the method further comprises disposing a second semipermeable ultrafiltration membrane on a second surface of the planar scaffold.

In certain aspects, the agarose is an ultra-low gelling agarose. In certain aspects, the agarose is present at a concentration of 1%-10% w/v, 2%-10% w/v, 2%-8% w/v, or 3%-6% w/v in the aqueous solution. In certain aspects, the dissolving the agarose comprises heating the aqueous solution to a temperature of about 37. C and stirring the solution at about 300 revolutions per minute (RPM). In certain aspects, creating an emulsion comprises stirring the solution at about 500-1000 RPM. In certain aspects, generating the matrix comprises cooling the emulsion to a temperature at which the agarose, gelatin, polyethylene glycol, PCL, collagen, alginate, dextran, or cellulose forms a gel.

In certain aspects, the biocompatible hydrophobic substance is a water-immiscible reagent, such as, perfluorodecalin (PFD). In certain aspects, an agarose solution, a PFD solution and a surfactant solution are combined at the about 63%-65% (v/v) agarose solution, about 32%-33% (v/v) PFD, and about 5%-2% (v/v) surfactant.

In certain aspects, the matrix may be washed with a solvent that dissolves the water-immiscible reagent to remove the water-immiscible reagent. In certain aspects, the matrix may be washed with an aqueous solution to remove the surfactant. In certain aspects, the matrix may be washed with mixture of a hydrophobic material and an aqueous solution to remove the water-immiscible reagent and the surfactant.

### EXPERIMENTAL

As can be appreciated from the disclosure provided above, the present disclosure has a wide variety of applications. Accordingly, the following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Those of skill in the art will readily recognize a variety of noncritical parameters that could be changed or modified to yield essentially similar results. Thus, the following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, dimensions, etc.) but some experimental errors and deviations should be accounted for.

### Materials and Methods

### SPA Scaffold Fabrication

Superporous agarose (SPA) scaffolds were constructed by dissolving ultra-low gelling agarose (Sigma-Aldrich: A2576) in 5 mL of Hank's Balanced Salt Solution (HBSS) (UCSF Cell Culture Facility: CCFAJ002) to create a 3% or 6% w/v solution in a beaker. The solution was further dissolved with five short cycles of heating in a microwave and gentle mixing between each cycle. The agarose solution was then heated on a hot plate to 37° C. and stirred at 300 revolutions per minute (RPM). Perfluorodecalin (PFD) (Sigma-Aldrich: P9900) and Tween™M 80 (Sigma-Aldrich: P4780) were then added to the agarose solution to create an emulsion consisting of agarose (64%, v/v), PFD (33%, v/v), and Tween™ 80 (3%, v/v). PFD was selected to substitute cyclohexane as the water-immiscible solvent due to its biocompatibility and similar cyclical structure [1]. The beaker was then sealed with Parafilm (Bemis Co, Inc), stirred for approximately 10 minutes at 750 RPM until thoroughly mixed, and emulsified, as indicated by its cloudy color.

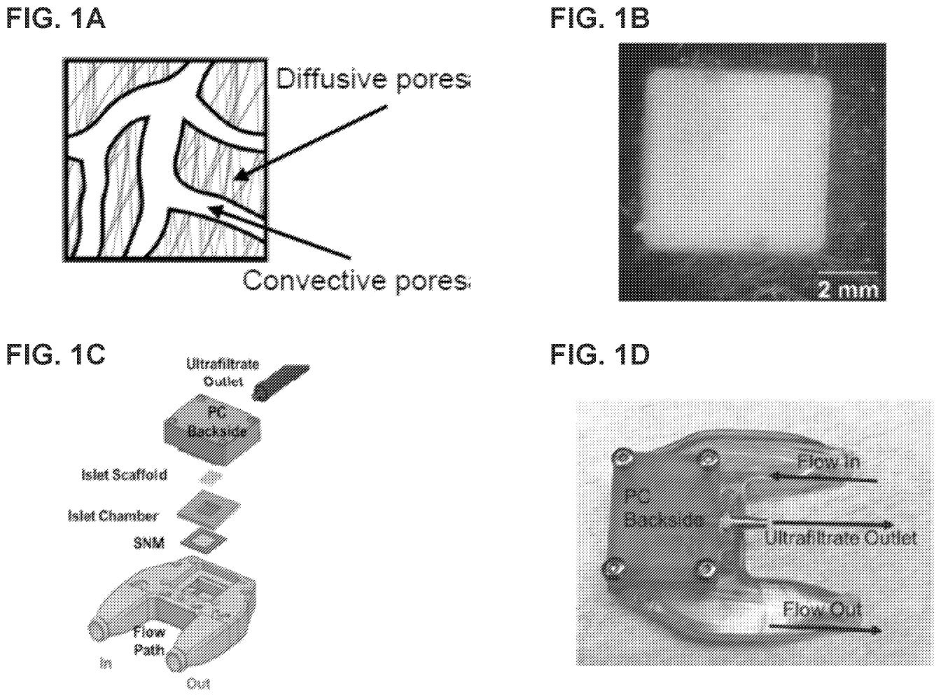

The fully mixed superporous agarose emulsion was then cast into a 316L stainless steel cell scaffold housing. If experiments included cells, 36 uL of the emulsion were mixed with the desired number of cells prior to casting in the cell scaffold housing. In order to achieve gelation, the scaffolds were placed at room temperature (25° C.) for 20 minutes followed by cooling at 4° C. for 10 minutes. Gelled scaffolds are comprised of convective pores created by the PFD droplets and diffusive pores that already exist within the native microstructure of the agarose (FIG. 1A). Scaffolds (FIG. 1B) were then inserted into intravascular bioartificial pancreas (iBAP) device (FIG. 1C). All in vitro testing used the iBAP device prototype previously developed by our group for islet encapsulation under convective mass transport (FIG. 1D) [2].

FIG. 1A-1D depict an SPA scaffold and intravascular bioartificial pancreas (iBAP) device assembly. FIG. 1A presents a schematic cross-sectional view of the SPA scaffold depicting the convective and diffusive pores in agarose. FIG. 1B presents an SPA scaffold within the islet chamber housing. FIG. 1C depicts an exploded view of the components of iBAP: flow path, silicon nanopore membrane (SNM), cell scaffold within the cell chamber, and polycarbonate (PC) backside and ultrafiltrate outlet. FIG. 1D presents an assembled iBAP used for in vitro testing.

### Hydraulic Permeability Measurement

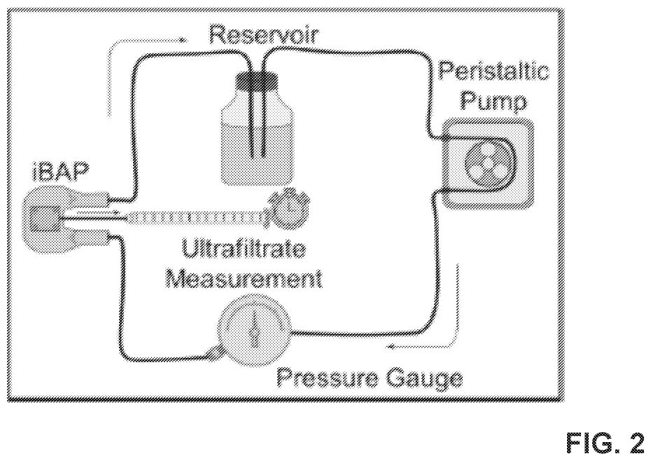

In order to test the hydraulic permeability of the SPA cell scaffold alone, the iBAP prototype was assembled without the SNM and connected to a custom flow circuit (FIG. 2). Masterflex L/S 25 tubing (Cole-Parmer) was connected to both the inlet and outlet of the device. A peristaltic pump (Cole-Parmer) controlled the crossflow rate in the device. A pressure gauge (General Electric) was placed in-line with the tubing to assess transmembrane pressure (TMP) across the scaffold. The ultrafiltrate outlet was connected to Masterflex L/S 14 tubing (Cole-Parmer). Ultrafiltrate rate was determined by the time required to produce 1 mL of ultrafiltrate, measured three times per scaffold. The TMP, ultrafiltrate rate (UF), and surface area (SA) were used to calculate hydraulic permeability (Lp) using an equation derived from Darcy's Law (Eq. 1).

$$Lp = \frac{UF}{TMP * SA} \qquad ( 1 )$$

FIG. 2 depicts a hydraulic permeability testing setup consisting of the iBAP, a peristaltic pump, and a pressure gauge. Ultrafiltration measurements were determined using a graduated syringe and a timer.

### Droplet Image Acquisition and Analysis

SPA scaffolds used for imaging were prepared with 3% (w/v) agarose solution in the same manner as previously described. After gelation, (n=3) scaffolds were fixed in 10% formalin to prevent bacteria growth and placed on glass bottom microwell dishes (MatTek: P35G-1.5-14-C). Scaffolds were imaged with a differential interference contrast (DIC) 20× lens using a time-lapse wide-field microscope (Nikon Instruments). Z-stack images were taken at three different locations on the scaffolds and analyzed using FIJI (ImageJ) software. The Hough Circle Transform plugin (UCB Vision Sciences) was used on three slices per stack (n=9 images per scaffold) to determine PFD droplet sizes within the scaffolds. Results for droplet size were then used to calculate the relative droplet area by determining the area of the droplets and then dividing by the total area of the image. To visualize variation between SPA batches, the results were averaged for droplet size and relative droplet area (%) per scaffold.

### Degradation Study

SPA (n=15) scaffolds made with 3% (w/v) agarose solution were used for testing degradation in static conditions for 28 days. Scaffolds were placed in 12-well Transwell dishes (Millipore Sigma: PIXP01250) with 1 mL of FBS (Corning: 35011CV) supplemented with 10% Penicillin-Streptomycin (UCSF Cell Culture Facility: CCFGK004). Scaffolds were maintained at 5% CO2 and 37° C. for the duration of the experiment and FBS was changed in the wells every three to four days. Three scaffolds were removed from the culture dish every seven days for weighing while still hydrated.

### Cell Procurement, Culture, and Encapsulation

Adult human islets were obtained from both the UCSF Islet and Cellular Production Facility (Mission Center Building, San Francisco, CA) and Prodo Laboratories, Inc (Aliso Viejo, CA). Islets isolated from UCSF were transported in CMRL 1066 medium (Corning: 15110CV) within 24 hours of isolation. Islets from Prodo Labs were washed and incubated with PIM(s) media (Prodo Laboratories, Inc). The enriched β-clusters (eBCs) were produced as previously described [3]. Briefly, the MEL-1 INSGFP/w hPSCs were cultured with differentiation-inducing factors in a stepwise fashion recapitulating pancreas development consisting of 6 stages over 20 days. Cells were then sorted by fluorescence-activated cell sorting (FACS), and immature β-like cells were reaggregated and further cultured to form the eBCs. The eBCs were ready for functional and physiological studies by day 28. The eBCs were transported in GN9 medium (CMRL media supplemented with 1% Penicillin-Streptomycin, 10% FBS, 1:100 glutamax, 1:100 NEAA, 10 μM Alki II, 0.5 mM VitC, 1 μM T3, 1 mM cysteine, 10 μM zinc, and 10 μg/ml heparin). Cells from all sources were maintained overnight in a non-treated T75 flask at 5% CO2 and 37° C. prior to encapsulation. Cells, either 500 IEQ or 500 eBCs (approximately 1000 cells per cluster with an average diameter of 100 μm) in 36 μL of SPA, were added to 3% SPA emulsion after the mixing step and allowed to gel as described earlier.

### Viability and Histology

Cell survival was assessed immediately following encapsulation. After gelation, scaffolds were placed in glass bottom microwell dishes (MatTek: P35G-1.5-14-C) for confocal imaging. Scaffolds were incubated at room temperature with a live/dead stain (Invitrogen: L3224) for 15 minutes and washed three times with PBS prior to imaging with a fluorescence spinning-disk confocal microscope (Nikon Instruments). Viability was quantified using FIJI (ImageJ) software by subtracting the area of dead cells from the total cell area and then dividing by the total cell area [4]. Viability was averaged for human islets (n=11) and eBCs (n=7) in the SPA scaffold, where n refers to the number of clusters analyzed. A non-emulsified 3% agarose was used as a comparison with both human islets (n=9) and eBCs (n=5).

Scaffolds were fixed with 4% paraformaldehyde and processed at the Gladstone Institute (San Francisco, CA). The scaffolds were embedded in paraffin prior to sectioning. Five-micron thick sections were stained with hematoxylin and eosin (H&E). Images were taken on a light microscope (Leica) at 20× magnification. The images of the islets and scaffold material were enhanced by increasing the gamma to 1.3 and outlining the pore regions in FIJI.

### Glucose-stimulated Insulin Secretion Assay In Vitro

The dynamic glucose-stimulated insulin secretion (GSIS) assay used a closed mock-loop circuit that contained either CMRL media (supplemented with 10% fetal bovine serum (FBS) v/v) for human islets or GN9 media for eBCs in a 37° C. 5% CO₂-humidified incubator. Masterflex L/S 14 and 25 tubing (Cole-Parmer) were connected to the ultrafiltrate outlets and both the inlet and outlet of the iBAP device, respectively. A peristaltic pump (Cole-Parmer) maintained a constant flow through the scaffold within the iBAP device to produce an ultrafiltrate rate of 50 μL/min. The cells were stabilized for 120 minutes in low glucose (5 mM) medium before conducting the GSIS test. Ultrafiltrate samples were collected over the course of 16 minutes during the first low glucose (5 mM) phase. The glucose concentration was then increased (28 mM) by adding glucose to the media and ultrafiltrate collection continued for 30 minutes. The choice of 28 mM for glucose stimulation is based on a protocol (SOP Document: 3104, A03) from National Institute of Allergy and Infectious Diseases (NIAID) [5]. The glucose concentration was subsequently reduced to basal concentrations and samples were collected for 44 minutes. Ultrafiltrate samples were kept at −20° C. until they could be analyzed for insulin concentration. The secreted insulin concentration in the ultrafiltrate samples from the GSIS experiment was determined using an enzyme-linked immunosorbent assay (ELISA) kit (Mercodia: 10-1113-01). The product of insulin concentration (pg/L) and ultrafiltration rate (mL/min), normalized to the number of islets or eBCs, was used to calculate insulin production (pg/min/IEQ). Stimulation index (SI) was calculated by dividing the first phase insulin production by the averaged basal insulin production.

### Statistical Methods

The data were expressed as mean±SD for hydraulic permeability, relative droplet area, degradation and viability experiments. Statistical evaluations of the means were assessed with different tests using GraphPad Prism version 8.2. Hydraulic permeability and viability data were analyzed with a two-way analysis of variance (ANOVA), and relative droplet area and degradation were assessed with a one-way ANOVA. Post hoc comparisons were carried out using a Tukey's multiple comparisons test. A p-value of <0.05 was accepted as statistically significant for all analyses.

### RESULTS

### Hydraulic Permeability

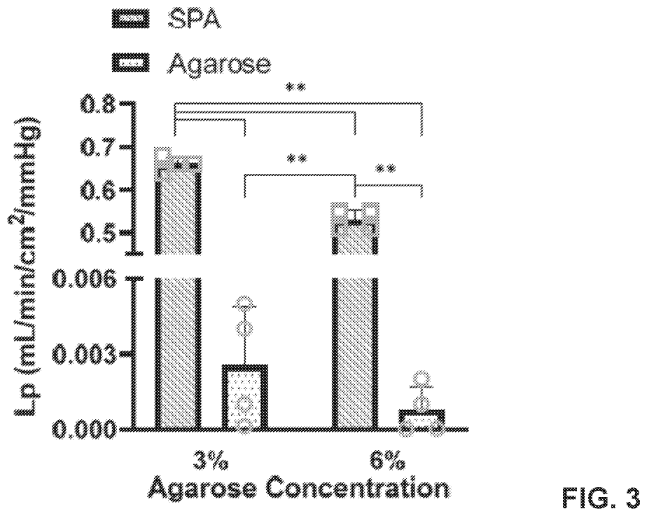

The optimization experiments compared the hydraulic permeability (Lp) of SPA scaffolds and non-emulsified agarose that were made with 3% (w/v) and 6% (w/v) agarose concentrations (FIG. 3). A two-way ANOVA found a significant interaction between the effects of agarose concentration (3% and 6%) and whether the scaffold was made with SPA or non-emulsified agarose on the hydraulic permeability (F(1,12)-81.6, p<0.001). SPA had a higher hydraulic permeability than non-emulsified agarose (F(1,12)-6986, p<0.001) for both agarose concentrations. Additionally, 3% SPA and 3% agarose had higher hydraulic permeability compared to their 6% counterparts (F(1,12)=86.1, p<0.001). Multiple comparison test showed significant differences between all groups (p<0.001) except scaffolds made with 3% and 6% non-emulsified agarose (0.0025 and 0.0008 mL/min/cm²/mmHg, respectively). The 3% SPA scaffold (n=4) had the highest hydraulic permeability and was significantly different (p<0.001) than the 6% SPA scaffold (n=4) (0.66 and 0.53 mL/min/cm^{2/}mmHg, respectively).

FIG. 3 shows that hydraulic permeability values (mean±SD, ** represents p<0.001) of scaffolds made with SPA and non-emulsified agarose were found to be statistically significant, F(1,12)-6986. There was also a significant effect F(1,12)-86.14, of agarose concentration (3% vs 6%). The post hoc test showed significant differences between all groups except the scaffolds made with 3% and 6% non-emulsified agarose (p=0.998). Hydraulic permeability was significantly different in the scaffold made with 3% (w/v) SPA than the other three scaffolds.

### PFD Droplet Analysis

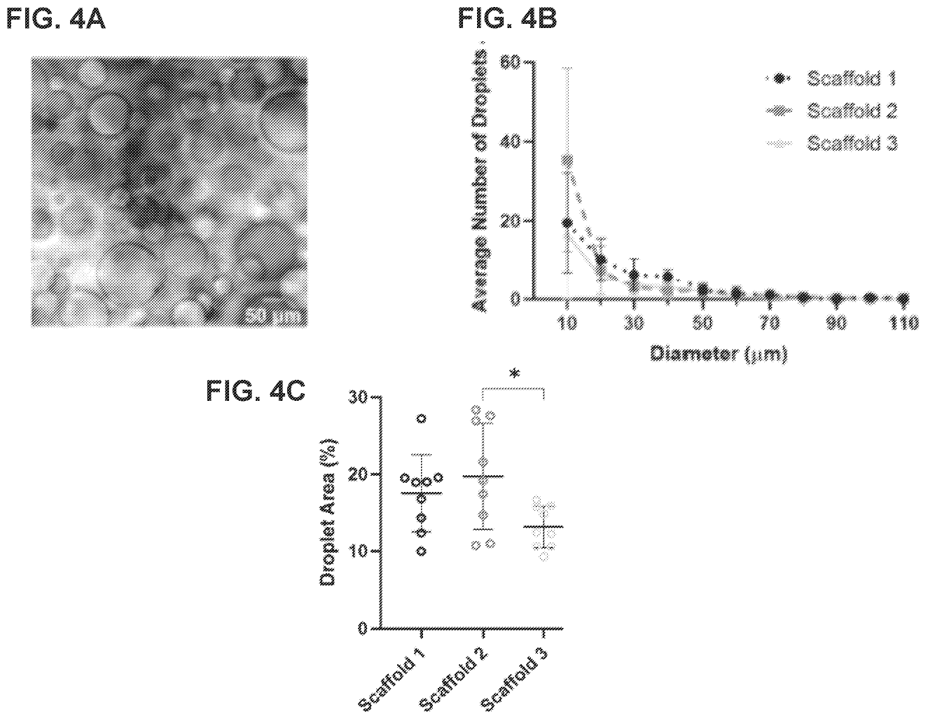

Three 3% SPA scaffolds were analyzed for PFD droplet size to compare variation between SPA batches (FIG. 4A). For every scaffold, z-stack images were obtained at three locations and at three different heights totaling nine DIC images per scaffold (n=9). PFD droplets were well dispersed and displayed no visible trend in placement within the scaffolds. Distribution of the PFD droplet sizes for each scaffold were markedly skewed towards smaller diameters (FIG. 4B). The median diameters of the PFD droplets were 18.2, 13, and 14.3 μm for three scaffolds, respectively. Relative droplet area of the scaffolds was calculated for each DIC image and then averaged (FIG. 4C). A one-way ANOVA was performed to compare the average droplet area (17.6±5.0, 19.7±6.9, and 13.2±2.7%, respectively) in the different scaffold batches and found a significant difference between the means (F(2,24)=3.8, p-0.037). Multiple comparisons test only found a significant difference between scaffolds 2 and 3 (p<0.05), indicating some variation between scaffolds.

FIG. 4A-4B depicts a PFD droplet size analysis. FIG. 4A depicts representative DIC images of the 3% SPA scaffold that was used for droplet analysis. FIG. 4B presents a histogram showing the size distribution of PFD droplets representative PFD droplets represented as mean±SD for each scaffold. FIG. 4C depicts the relative droplet area of the scaffold calculated for each DIC image and then averaged. Lines represent the mean±SD for each scaffold and * represents p<0.05.

### Scaffold Degradation Study

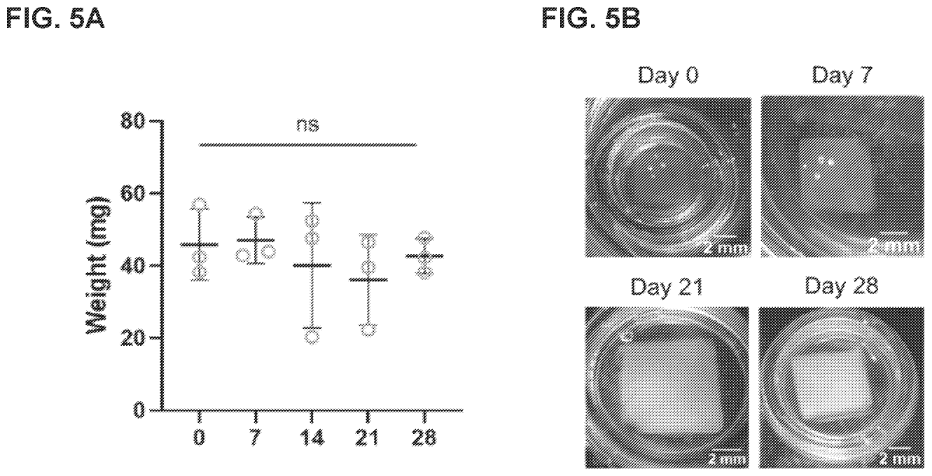

The change in hydrated scaffold weight was assessed over 28 days in static culture conditions. The average weight of the day 0 scaffolds was 45.8±9.78 mg, while the average weight of the day 28 scaffolds was 42.7±4.81 mg (FIG. 5A). A one-way ANOVA determined there was no significant change in the average weights of the scaffolds over time (F(4,10)=0.048, p=0.75). However, appearance of the scaffolds changed over the 28-day study. Scaffolds became less translucent after 21 days and looked almost white in color by day 28 (FIG. 5B) potentially due to protein deposition from the FBS in the media.

FIG. 5A-5B depict the degradation of the 3% SPA scaffolds. FIG. 5A show that the 28-day degradation study measured changes in weight and found no significant difference at the different time points. Data is shown as mean±SD. FIG. 5B shows representative images of the scaffold at 0, 7, 21, and 28 days of culture.

### Histology and Cell Viability Assessment

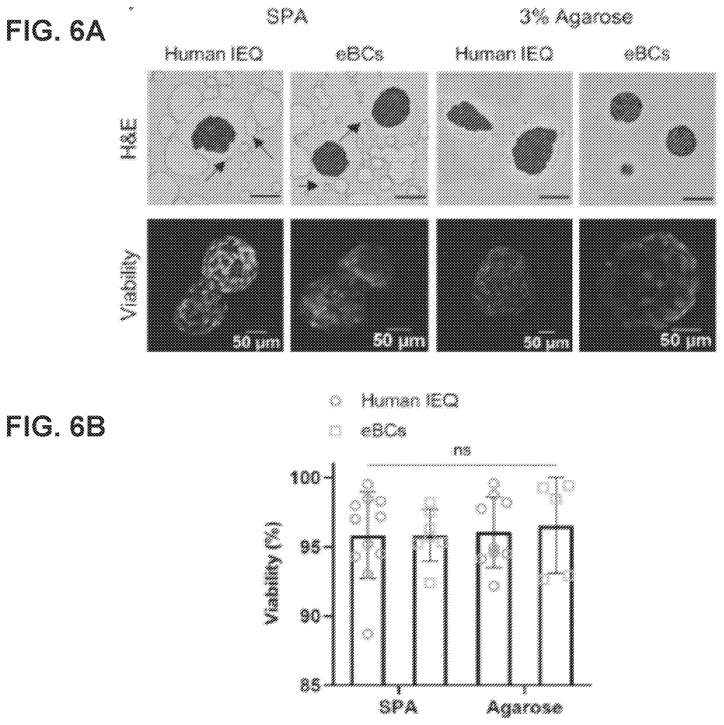

To visualize the morphology of both the cells and the scaffolds, histological sections were stained with H&E (FIG. 6A, top). Human islets and eBCs in both SPA and traditional agarose display similar morphology. SPA sections display open areas (pores) within the scaffold indicated by the dotted outlines. The pores appear to be distributed throughout the scaffold and nearby (<10 μm, FIG. 6A, top) the encapsulated cells.

Adult human islet and eBC viability was measured immediately after encapsulation within both the optimized SPA formulation and traditional, non-emulsified agarose (FIG. 6A, bottom). Multiple cell clusters were imaged for both human islets and eBCs (FIG. 6B). The human islets were 95.8±3% viable (n=11), and the eBCs were 95.8±2% viable (n=7) in the SPA scaffolds. In the non-emulsified 3% agarose scaffolds, human islets and eBCs were 96.1±3% (n=9) and 96.6±3% (n=5) viable. Dead cells were mostly observed on the periphery of the islets. For the eBCs, dead cells were seen mostly in periphery, while a few were observed in the center of the cluster in the SPA scaffold. A two-way ANOVA determined there was no significant difference in average viability between the different scaffolds (F(1,28)-0.2109, p=0.65), nor the different cell types (F(1,28)-0.050, p=0.83). Overall, the high viability demonstrated that the encapsulation process did not damage the cells, allowing for further dynamic glucose-stimulated insulin secretion evaluation.

FIG. 6A-6B presents a histologic evaluation and viability of human islets and eBCs in the 3% traditional agarose and SPA scaffolds. FIG. 6A depicts representative images from H&E and viability staining of human islets and eBCs in traditional agarose and SPA scaffolds. H&E staining shows nuclei and cytoplasm, and the dotted lines indicate the pores (arrows) surrounding the encapsulated cell clusters in SPA. Scale bars represent 50 μm in length. Viability staining shows dead cells and live cells, which were imaged with a fluorescence microscope. FIG. 6B presents results for viability reported as mean+SD. Human islets were 95.8±3% viable in the SPA scaffold and 96.6±3% viable in the traditional agarose scaffold. eBCs were 95.8±2% viable in the SPA scaffolds and 96.6±3% viable in the traditional agarose scaffold. Two-way ANOVA determined there was no significant difference between the mean viability of the groups.

### Assessment of Insulin Production

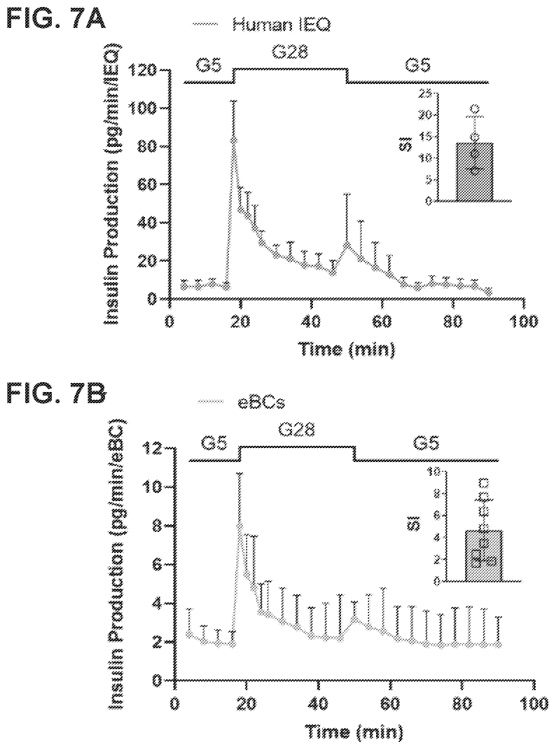

The SPA scaffolds containing either human islets (n=4) or eBCs (n=8) with 500 IEQ or eBCs in 36 μL of SPA were tested in the iBAP using an in vitro GSIS assay. Average insulin production during the first low glucose exposure was 6.8±2.7 pg/min/IEQ for human islets (FIG. 7A) and 2.1±0.9 pg/min/eBC for eBCs (FIG. 7B). When exposed to higher levels of glucose (28 mM), both adult human islets and eBCs increased insulin production and demonstrated a biphasic insulin response. First phase insulin production for human islets was 83.4±20.5 pg/min/IEQ, and 8.0±2.7 pg/min/eBC for eBCs after exposure to a glucose concentration of 28 mM. During the second phase, insulin production gradually decreased from the first phase peak with an average insulin production of 27.8±14.0 pg/min/IEQ and 3.3±2.1 pg/min/eBC for human islets and eBCs, respectively. All scaffolds sustained insulin production above baseline insulin levels during the second phase and showed a small spike in insulin production after second low glucose exposure. During the second low glucose exposure, basal insulin production returned to pre-high glucose stimulatory levels (6.4±3.3 pg/min/IEQ and 2.0±1.6 pg/min/eBC). The stimulation index (SI), or the ratio of first phase insulin production during high glucose exposure to basal insulin production, was 13.6±6.1 in human islets and 4.9±3.4 in eBCs.

FIG. 7A-7B depicts an in vitro assessment of human islet and eBC insulin production in the optimized SPA formulation. Insulin production in the SPA scaffold is shown for both human islets (n=4) in FIG. 7A and eBCs (n=8) in FIG. 7B during the GSIS exposure to 5mM of glucose (G5), 28 mM of glucose (G28), followed by a second G5 phase. Data represents mean±SD.

### Discussion

A novel superporous agarose-based cell scaffold was fabricated that supported eBC and islet viability and insulin production within the iBAP. This scaffold aims to optimize mass transfer of key solutes to and from the islets in two ways. First, the highly porous (superporous) structure enables permeation of ultrafiltrate throughout the scaffold therefore minimizing the diffusion distance (<10 μm) from ultrafiltrate to islets or eBCs, as demonstrated by the hydraulic permeability and porosity experiments. Second, the scaffold's high hydraulic permeability does not restrict flow of the SNM-generated ultrafiltrate through the cell scaffold. Most polymer membranes used for ultrafiltration exhibit insufficient hydraulic permeability, due to the membrane thickness (30-100 μm), necessary to sustain islet viability and insulin production [6]. Unlike conventional polymer membranes, SNM are less than 1 μm-thick with a highly uniform pore size distribution which enables higher amounts of ultrafiltrate and could improve islet functionality [6, 7, 2]. For the iBAP design to deliver adequate amounts of ultrafiltrate to the encapsulated islets, the hydraulic permeability of the cell scaffold must be greater than the SNM. High agarose concentration (6% w/v) is widely used and favored in bioseparation and chromatography processes because of its small pore size [8]. The small pore size in high agarose concentrations are known to create e a rigid microenvironment, which can initiate mechanotransduction in encapsulated islets [9]. Also, the small pore size in high agarose concentrations can limit hydraulic permeability [10], which is consistent with our finding that the 3% (w/v) SPA scaffolds had significantly greater hydraulic permeability than the 6% (w/v) SPA scaffolds. SPA scaffolds made with 3% (w/v) agarose have a 70-fold greater hydraulic permeability than the SNM (0.66 and 0.010 mL/min/cm²/mmHg, respectively) [7]. Hydraulic permeability is inversely related to resistance to fluid flow, and hence, the high hydraulic permeability of the scaffolds results in lower resistance to fluid flow than the SNM. To maximize ultrafiltrate rate in the iBAP, the overall hydraulic permeability (Lₒ) can be calculated (Eqn 2) by taking the inverse sum of the hydraulic permeabilities of the cell scaffold (Lₚ) and the SNM (L_{SNM}) [11].

$$\frac{1}{{L}_{o}} = \frac{1}{{L}_{p}} + \frac{1}{{L}_{SNM}} \qquad ( 2 )$$

Therefore, the controlling hydraulic resistance will be the SNM and the ultrafiltrate rate will be predominately dependent on the hydraulic resistance of the SNM. Consequently, the use of SPA scaffold will not decrease the overall ultrafiltration rate through the iBAP.

Human islets demonstrated dynamic insulin production in response to glucose stimulation in the SPA scaffold. The baseline insulin production rate during the first low glucose exposure was 6-7 pg/min/IEQ and consistent with other human islet studies in vitro at ˜5 mM glucose concentrations [12]. Studies using perifused human islets have shown insulin production peaks from 12-40 pg/min/IEQ when exposed to 16.7 mM glucose [12-14], while the SPA scaffolds demonstrated a higher production (60-99 pg/min/IEQ) at 28 mM glucose. We chose a concentration of 28 mM for glucose stimulation as it is traditionally used in static GSIS assays for clinical transplantation assessment [5]. While the distinct biphasic curve in response to glucose stimulation shows promise for insulin therapy, long-term assessments of insulin response and diabetic porcine studies will be needed for confirmation. These studies demonstrated initial feasibility and a biphasic response to stimulation with high glucose with a distinct first phase and a shutdown of insulin production following a return to low glucose levels, indicating physiologic islet function in the SPA scaffold in vitro.

Recently, advances have been made in the formation of β cells from hPSCs to improve insulin secretion so that the cells display responses similar to mature β cells [3, 15, 16]; however, integration of these cells in transplantable devices is still in infancy. There are groups exploring BAP devices with previous generations of hPSC-derived cells conducting preclinical studies and undergoing clinical trials, such as Semma Therapeutics (subsidiary of Vertex Pharmaceuticals, Boston, MA) and ViaCyte (San Diego, CA) [17-20]. It was found that human islets and eBCs exhibited similar insulin response profiles to glucose stimulation in the SPA scaffold. It is believed that this study is the first to demonstrate dynamic insulin secretory kinetics with stem-cell-derived beta cells in a macroencapsulation device in vitro. The first phase began at the first time point of the high glucose challenge for both cell types which demonstrates no delay in glucose-insulin kinetics [21]. The magnitude of insulin secretion was considerably smaller in the eBCs than the human islets, a finding that is consistent with many other hPSCs-derived β cell studies [22-24] in addition to the fact that the MEL-1 INSGFP/w line used in our study carries only one functional insulin allele [23]. The other insulin allele in the eBCs is replaced by GFP for sorting purposes [3], and restoring the second insulin allele could theoretically double the insulin production. Additionally, a second insulin production peak was observed after exposure to the second low glucose concentration medium in both eBCs and human islets. Henquin et al. made a similar observation, called the off-response, in perifused infant and adult human islets where insulin would peak prior to returning to basal levels [25]. Other reports have shown that hPSC-derived β cells fail to terminate the insulin response to decreased glucose concentrations, which is a characteristic of immature β cells [19, 26, 27]. The proper termination of elevated insulin production in the eBCs indicates that they function similarly to adult human islets and can prevent hypoglycemia [15, 28]. While the overall insulin production was lower in the eBCs, the optimal mass transport in the SPA scaffold indicated the promise of supporting larger cell densities while maintaining a normoxic environment in the iBAP. In summary, the present findings demonstrated, for the first time, that eBCs have similar insulin response profiles to glucose stimulation as adult human islets in the SPA scaffold.

The SPA macroporous scaffold within the iBAP appeared to provide adequate mass transfer to macroencapsulated insulin-producing cells by minimizing the diffusion distance between the ultrafiltrate and islets. Previous reports of the SNM-based iBAP have shown that ultrafiltrate-mediated, convection-based mass transfer across the SNM and through the agarose cell scaffold supports islet function [2]. Although traditional agarose has been widely used for islet encapsulation [29-31], it does not allow for high flux of solutes. Further characterization of the SPA scaffold indicated high porosity based on relative PFD droplet area (>13.2±2.7%) and minimal degradation over 28 days. It is currently unknown how much PFD remains as droplets and how much is removed to create open pores. The apparent color change in the SPA scaffolds could either be due to protein or albumin deposition from the FBS Another hypothesis includes increasing emulsion instability overt time. Ongoing fluorine nuclear magnetic resonance (fNMR) spectroscopy studies will help determine PFD levels over time within SPA scaffolds. Histologic evaluation of the SPA scaffold revealed pores surrounding the outer surface of islet (<10 μm from the pore to the islet surface), enabling ultrafiltrate to flow nearly directly across the islet's surface. Consequently, the diffusion distance from the ultrafiltrate to the islet's center is defined by the islet's diameter. Additionally, changing the scaffold from agarose to SPA increased the hydraulic permeability, or fluid velocity, therefore increasing the Reynold's number which will improve the overall mass transfer within the scaffold [11, 32]. The short diffusion distance and increased hydraulic permeability significantly increases the transport of oxygen, glucose, and insulin within the cell scaffold. A SPA cell scaffold for islet encapsulation is ideal for maintaining normoxic conditions and providing fast glucose-insulin kinetics for both human islets and stem-cell derived eBCs within a convection-based device, demonstrating its potential application in an intravascular bioartificial pancreas.

### REFERENCES

1. Lowe K C. Second-Generation Perfluorocarbon Emulsion Blood Substitutes. Artificial Cells, Blood Substitutes, and Biotechnology 2000; 28: 25-38.
2. Song S, Blaha C, Moses W, et al. An intravascular bioartificial pancreas device (iBAP) with silicon nanopore membranes (SNM) for islet encapsulation under convective mass transport. Lab Chip 2017; 17:1778-1792.
3. Nair G G, Liu J S, Russ H A, et al. Recapitulating endocrine cell clustering in culture promotes maturation of human stem-cell-derived β cells. Nat Cell Biol 2019; 21: 263-274.
4. Schindelin J, Arganda-Carreras I, Frise E, et al. Fiji: an open-source platform for biological-image analysis. Nat Methods 2012; 9: 676-682.
5. Ricordi C, Goldstein J S, Balamurugan A N, et al. National Institutes of Health-Sponsored Clinical Islet Transplantation Consortium Phase 3 Trial: Manufacture of a Complex Cellular Product at Eight Processing Facilities. 2016; 65: 11.
6. Song S, Roy S. Progress and challenges in macroencapsulation approaches for type 1 diabetes (T1D) treatment: Cells, biomaterials, and devices: Progress and Challenges in Macroencapsulation. Biotechnol Bioeng 2016; 113: 1381-1402.
7. Song S, Faleo G, Yeung R, et al. Silicon nanopore membrane (SNM) for islet encapsulation and immunoisolation under convective transport. Sci Rep 2016; 6: 23679.
8. Gustavsson P-E, Tiainen P, Larsson P-O. Superporous Agarose Gels: Production, Properties, and Applications. In: Mattiasson B, Kumar A, Galaev I (eds) Macroporous Polymers. CRC Press, pp. 155-178.
9. Barkai U, Rotem A, de Vos P. Survival of encapsulated islets: More than a membrane story. WJT 2016; 6:69.
10. Johnson E M, Deen W M. Hydraulic permeability of agarose gels. AIChE J 1996; 42: 1220-1224.
11. Basic Transport Phenomena in Biomedical Engineering, Fourth Edition. CRC Press. Epub ahead of print 7 Aug. 2017. DOI: 10.1201/9781315120478.
12. Fujita Y, Takita M, Shimoda M, et al. Large human islets secrete less insulin per islet equivalent than smaller islets in vitro. Islets 2011; 3: 1-5.
13. Dai C, Brissova M, Hang Y, et al. Islet-enriched gene expression and glucose-induced insulin secretion in human and mouse islets. Diabetologia 2012; 55: 707-718.
14. Lehmann R, Zuellig R A, Kugelmeier P, et al. Superiority of Small Islets in Human Islet Transplantation. Diabetes 2007; 56: 594-603.
15. Zhang D, Jiang W, Liu M, et al. Highly efficient differentiation of human ES cells and iPS cells into mature pancreatic insulin-producing cells. Cell Res 2009; 19: 429-438.
16. Hogrebe N J, Augsornworawat P, Maxwell K G, et al. Targeting the cytoskeleton to direct pancreatic differentiation of human pluripotent stem cells. Nature Biotechnology 2020; 38: 460-470.
17. Vegas A J, Veiseh O, Gürtler M, et al. Long-term glycemic control using polymer-encapsulated human stem cell-derived beta cells in immune-competent mice. Nat Med 2016; 22: 306-311.
18. Pullen L C. Stem Cell-Derived Pancreatic Progenitor Cells Have Now Been Transplanted into Patients: Report from IPITA 2018. Am J Transplant 2018; 18: 1581-1582.
19. Pagliuca F W, Millman J R, Gürtler M, et al. Generation of Functional Human Pancreatic β Cells In Vitro. Cell 2014; 159: 428-439.
20. Jellali R, Essaouiba A, Leclerc E, et al. Membrane bioreactors for bio-artificial pancreas. In: Current Trends and Future Developments on (Bio-) Membranes. Elsevier, pp. 77-108.
21. Colton C K. Oxygen supply to encapsulated therapeutic cells. Advanced Drug Delivery Reviews 2014; 67-68: 93-110.
22. Massumi M, Pourasgari F, Nalla A, et al. An Abbreviated Protocol for In Vitro Generation of Functional Human Embryonic Stem Cell-Derived Beta-Like Cells. PLOS ONE 2016; 11: e0164457.
23. Velazco-Cruz L, Song J, Maxwell K G, et al. Acquisition of Dynamic Function in Human Stem Cell-Derived β Cells. Stem Cell Reports 2019; 12: 351-365.
24 Micallef S J, Li X, Schiesser J V, et al. INS(GFP/w) human embryonic stem cells facilitate isolation of in vitro derived insulin-producing cells. Diabetologia 2012; 55: 694-706.
25. Henquin J-C, Nenquin M. Dynamics and Regulation of Insulin Secretion in Pancreatic Islets from Normal Young Children. PLOS ONE 2016; 11: e0165961.
26. Rezania A, Bruin J E, Arora P, et al. Reversal of diabetes with insulin-producing cells derived in vitro from human pluripotent stem cells. Nat Biotechnol 2014; 32: 1121-1133.
27. Russ H A, Parent A V, Ringler J J, et al. Controlled induction of human pancreatic progenitors produces functional beta-like cells in vitro. EMBO J 2015; 34: 1759-1772.
28. Hrvatin S, O'Donnell C W, Deng F, et al. Differentiated human stem cells resemble fetal, not adult, β cells. Proc Natl Acad Sci USA 2014; 111: 3038-3043.
29. Gin H, Dupuy B, Baquey C, et al. Agarose encapsulation of islets of Langerhans: reduced toxicity in vitro. Journal of Microencapsulation 1987; 4: 239-242.
30. Dimitrioglou N, Kanelli M, Papageorgiou E, et al. Paving the way for successful islet encapsulation. Drug Discovery Today 2019; 24: 737-748.
31. Teramura Y, Iwata H. Bioartificial pancreas microencapsulation and conformal coating of islet of Langerhans. Adv Drug Deliv Rev 2010; 62: 827-840.
32. Pradanos P, Arribas J I, Hernandez A. Hydraulic Permeability, Mass Transfer, and Retention of PEGs in Cross-flow Ultrafiltration through a Symmetric Microporous Membrane. Separation Science and Technology 1992; 27: 2121-2142.

1. A biocompatible gel matrix comprising:
a comprising a plurality of microchannels and a plurality of nanochannels, wherein the plurality of microchannels and the plurality of nanochannels are not patterned microchannels and nanochannels; and
a plurality of cells, wherein the cells are adjacent the plurality of microchannels and wherein a majority of the plurality of cells are within a distance of 50 microns or less from at least one of the plurality of microchannels,
wherein the plurality of microchannels have a width of 5-500 microns and the plurality of nanochannels have a width of 1 nm-500 nm.

2. The gel matrix of claim 1, wherein the gel matrix is composed of agarose.

3. The gel matrix of claim 1, wherein the gel matrix is composed of collagen, alginate, cellulose, polyethylene glycol, polycaprolactone (PCL), gelatin, or dextran.

4. The gel matrix of any one of claims 1-3, wherein the matrix is in form of a planar scaffold, a cylinder, a sphere, or fibers.

5. The gel matrix of any one of claims 1-4, wherein the microchannels allow flow of nutrients to the plurality of cells and wherein at least 80% of the plurality of cells encapsulated in the matrix are viable for at least 1 day.

6. The gel matrix of any one of claims 1-5, wherein the microchannels allow flow of nutrients to the plurality of cells and wherein at least 80% of the plurality of cells encapsulated in the matrix are viable for up to 1 month.

7. The gel matrix of any one of claims 1-4, wherein the microchannels allow flow of nutrients to the plurality of cells and wherein at least 80% of the plurality of cells encapsulated in the matrix are viable and functional for at least 1 day.

8. The gel matrix of any one of claims 1-4, wherein the microchannels allow flow of nutrients to the plurality of cells and wherein at least 80% of the plurality of cells encapsulated in the matrix are viable and functional for up to 1 month.

9. The gel matrix of any one of claims 1-8, wherein the plurality of cells are insulin producing cells.

10. The gel matrix of claim 9, wherein the insulin producing cells are derived from differentiation of stem cells.

11. The gel matrix of claim 9, wherein the insulin producing cells are pancreatic cells isolated from pancreatic islets.

12. The gel matrix of claim 9, wherein the insulin producing cells are in islets isolated from pancreas and the islets are encapsulated in the matrix.

13. The gel matrix of claim 12, wherein the islets each comprises about 1000 cells.

14. The gel matrix of claim 12 or 13, wherein each islet has a diameter of about 100 microns.

15. The gel matrix of claim 9, wherein the insulin producing cells are in stem-cell-derived enriched β-clusters (eBCs).

16. The gel matrix of claim 15, wherein each eBC comprises about 1000 cells.

17. The gel matrix of claim 15 or 16, wherein each eBC has a diameter of about 100 microns.

18. A bioartificial ultrafiltration device comprising:
a planar scaffold comprising a matrix of any one of claims 1-17;
a first semipermeable ultrafiltration membrane disposed on a first surface of the planar scaffold;
a first compartment adjacent to the first surface of the planar scaffold and in fluidic communication with the planar scaffold via the first semipermeable ultrafiltration membrane and comprising an inlet and an outlet; and
a second compartment adjacent to the second surface of the planar scaffold and comprising an outlet,
wherein the first semipermeable ultrafiltration membrane comprises a plurality of pores having a width in the range of 5 nm-5 micron,
wherein the first semipermeable ultrafiltration membrane allows transport of ultrafiltrate from the first compartment to the matrix and wherein the ultrafiltrate traverses through the matrix into the second compartment.

19. The device of claim 18, wherein the device further comprises a second semipermeable ultrafiltration membrane disposed on the second surface of the planar scaffold and wherein the ultrafiltrate traverses from the plurality of microchannels across the second semipermeable ultrafiltration membrane into the second compartment.

20. The device of claim 19, wherein the second semipermeable ultrafiltration membrane comprises a plurality of pores having a width in the range of 5 nm-5 micron.

21. The device of claim 19 or 20, wherein the first and second semipermeable ultrafiltration membranes comprise a plurality of pores having a width in the range the range of 0.1 microns -2 microns.

22. The device of any one of claims 19-21, wherein the second semipermeable ultrafiltration membrane comprises a plurality of pores having a width larger than the width of the plurality of pores in the first semipermeable ultrafiltration membrane.

23. The device of any one of claims 18-22, wherein the inlet of the first compartment is attachable to a tubing for connection to a blood vessel of a subject, optionally, wherein the blood vessel is an artery of the subject.

24. The device of any one of claims 18-23, wherein the outlet of the first compartment is attachable to a tubing for connection to a blood vessel of a subject, optionally, wherein the blood vessel is a vein of the subject or to an artery of the subject.

25. The device of any one of claims 18-24, wherein the outlet of the second compartment is attachable to a tubing for connection to (i) a blood vessel of a subject, and optionally provides the ultrafiltrate to one or more blood vessels of the subject, (ii) one or more veins of the subject, (iii) one or more arteries of the subject; and/or (iv) to an analyte analysis device.

26. The device of any one of claims 18-25, wherein the plurality of pores in the first semipermeable membrane have a width in the range of 0.2 μm-0.5 μm, 20 nm-2 microns, or 20 nm-50 nm.

27. The device of any one of claims 19-26, wherein the plurality of pores in the second semipermeable membrane have a width in the range of 0.2 μm-0.5 μm, 20 nm-2 microns, or 20 nm-50 nm.

28. The device of any one of claims 18-27, wherein the thickness of the first semipermeable ultrafiltration membrane is in the range of 0.1 micron-100 micron, 0.5 μm-10 μm.

29. The device of any one of claims 19-28, wherein the thickness of the second semipermeable ultrafiltration membrane is in the range of 0.1 micron-100 micron or 0.5 μm-10 μm.

30. The device of any one of claims 18-29, the surface of the first and/or the second surface of the planar scaffold is in the range of 1 cm²-100 cm² or 15 cm²-30 cm².

31. The device of any one of claims 18-30, wherein the surface area of the first semipermeable ultrafiltration membrane is in the range of 1 cm²-1000 cm² or 15 cm²-30 cm².

32. The device of any one of claims 19-31, wherein the surface area of the second semipermeable ultrafiltration membrane is in the range of 1 cm²-1000 cm² or 15 cm²-30 cm².

33. The device of any one of claims 18-32, wherein the plurality of pores are circular in shape and wherein the width refers to diameter of the pores.

34. The device of any one of claims 18-33, wherein the plurality of pores are slit-shaped.

35. The device of any one of claims 18-34, wherein the plurality of pores are slit-shaped and wherein the width of the pores is 5 nm-100 nm.

36. The device of claim any one of claims 18-44, wherein the plurality of pores are slit-shaped and wherein the length of the pores is in the range of 0.1 micron-5 micron.

37. The device of claim any one of claims 18-36, wherein the plurality of pores are slit-shaped and wherein the length of the pores is in the range of 1 μm-3 μm.

38. The device of any one of claims 18-37, wherein the cells are autologous to the subject comprising the device.

39. The device of any one of claims 18-37, wherein the cells are xenogenic to the subject comprising the device.

40. The device of any one of claims 18-37, wherein the cells are allogenic to the subject comprising the device.

41. A bioartificial ultrafiltration device comprising:
a planar scaffold comprising the matrix of any one of claims 1-17;
a first semipermeable ultrafiltration membrane of any one of claims 18-40 disposed on a first surface and a second semipermeable ultrafiltration membrane of any one of claims 19-40 disposed on a second surface of the planar scaffold;
a first compartment comprising a first inlet and a first outlet, wherein the first compartment is adjacent to the first surface of the planar scaffold;
a second compartment comprising a second inlet and a second outlet, wherein the second compartment is adjacent to the second surface of the planar scaffold,
wherein the first inlet is configured for connection to an artery of a subject and the first outlet is connected to the second inlet of the second compartment,
wherein the second outlet of the second compartment is configured for connection to a vein of the subject,
wherein the semipermeable ultrafiltration membranes comprise a plurality of pores having a width in the range of 5nm-5 micron,
wherein the first semipermeable ultrafiltration membrane allows transport of ultrafiltrate from the first compartment to the scaffold and the second semipermeable ultrafiltration membrane allows transport of the ultrafiltrate from the plurality of microchannels in the scaffold into the second compartment.

42. The device of claim 41, wherein the cells are autologous to the subject.

43. The device of claim 41, wherein the cells are xenogenic to the subject.

44. The device of claim 41, wherein the cells are allogenic to the subject.

45. The device of any one of claims 41-44, wherein the plurality of pores in the second semipermeable ultrafiltration membrane have a width larger than the width of the plurality of pores in the first semipermeable ultrafiltration membrane or wherein the plurality of pores in the second semipermeable ultrafiltration membrane have a width smaller than the width of the plurality of pores in the first semipermeable ultrafiltration membrane.

46. A method for providing a bioartificial ultrafiltration device comprising cells to a subject in need thereof, the method comprising:
connecting the bioartificial ultrafiltration device of any one of claims 18-40 to the subject, wherein the connecting comprises:
connecting the inlet of the first compartment to an artery of the subject and connecting the outlet of the first compartment to a blood vessel of the subject; and
connecting the outlet of the second compartment to a blood vessel or a body cavity of the subject; or
connecting the outlet of the second compartment to an analyte analysis device.

47. A method for providing a bioartificial ultrafiltration device comprising cells to a subject in need thereof, the method comprising:
connecting the bioartificial ultrafiltration device of any one of claims 41-45 to the subject, wherein the connecting comprises:
connecting the first inlet to an artery of a subject; and
connecting the second outlet to a vein of the subject.

48. The method of any one of claims 46-47, wherein the method comprises providing insulin to the subject and wherein the cells comprise insulin producing cells.

49. The method of any one of claims 46-48, wherein connecting the bioartificial device to the subject in need thereof results in increased viability of the cells in the scaffold.

50. The method of any one of claims 46-49, wherein the ultrafiltrate comprises one or more of glucose and oxygen.

51. The method of any one of claims 46-50, wherein the ultrafiltrate comprises one or more of glucose and oxygen and wherein the insulin producing cells excrete insulin in response to presence of glucose in the ultrafiltrate and wherein the plurality of microchannels transport the insulin to the second compartment.

52. The method of claim 51, wherein the excreted insulin is transported to the plurality of microchannels in the scaffold.

53. The method of any one of claims 46-52, wherein the semipermeable ultrafiltration membranes prevent the passage of immune system components into the scaffold.

54. The method of any one of claims 46-53, wherein the semipermeable ultrafiltration membranes prevents passage of antibodies into the scaffold.

55. The method of any one of claims 46-54, the semipermeable ultrafiltration membranes prevents passage of cytokines into the scaffold.

56. The method of any one of claims 46-55, the semipermeable ultrafiltration membranes prevents passage of TNF-α, IFN-γ, and/or IL-1β into the scaffold.

57. A method of making a matrix comprising agarose, collagen, gelatin, polyethylene glycol, PCL, alginate, dextran, or cellulose comprising a plurality of microchannels and a plurality of nanochannels, wherein the plurality of microchannels and the plurality of nanochannels are not patterned microchannels and nanochannels and wherein the plurality of microchannels have a width of 5-500 microns and the plurality of nanochannels have a width of 1 nm-500 nm, the method comprising:
generating an aqueous solution comprising dissolved agarose, gelatin, polyethylene glycol, PCL, collagen, alginate, dextran, or cellulose;
adding a water-immiscible reagent and a surfactant to the aqueous solution;
mixing the aqueous solution under conditions sufficient for generation of an emulsion comprising the dissolved agarose, gelatin, polyethylene glycol, PCL, collagen, alginate, dextran, or cellulose, water-immiscible reagent and surfactant; and
generating the matrix by placing the emulsion at a temperature sufficient to allow gelation of the agarose, gelatin, polyethylene glycol, PCL, collagen, alginate, dextran, or cellulose, thereby creating the matrix.

58. The method of claim 57, further comprising:
adding cells to the emulsion prior to the step of generating the matrix.

59. The method of claim 57 or 58, wherein generating the matrix comprises casting the emulsion in a mold comprising a planar surface, thereby creating a planar scaffold.

60. The method of any one of claims 59, wherein method comprises disposing a first semipermeable ultrafiltration membrane on a first surface of the planar scaffold.

61. The method of any one of claims 57-60, wherein method comprises disposing a second semipermeable ultrafiltration membrane on a second surface of the planar scaffold.

62. The method of any one of claims 57-61, wherein the agarose is an ultra-low gelling agarose.

63. The method of claim 62, wherein the agarose is present at a concentration of 1%-10% w/v, 2%-10% w/v, 2%-8% w/v, or 3%-6% w/v in the aqueous solution.

64. The method of any one of claims 57-63, wherein dissolving the agarose comprises heating the aqueous solution to a temperature of about 37. C and stirring the solution at about 300 revolutions per minute (RPM).

65. The method of any one of claims 57-64, wherein the water-immiscible reagent is perfluorodecalin (PFD).
